(19) United States
(12) Patent Application Publication
(10) Pub. No.: US 2001/0025031 A1
(43) Pub. Date: Sep. 27, 2001

(54) HFGAN72 receptor genomic DNA, variants thereof, and methods of use thereof in diagnostic applications

(76) Inventors: Catherine E. Ellis, Glassboro, NJ (US); Cheni Kwok, Hatfield Heath (GB); Nicola J. Bodsworth, Walden (GB); Wendy Halsey, Kennett Square, PA (US); Stephanie Van Horn, Pottstown, PA (US)

Correspondence Address: Ratner & Prestia, P.O. Box 980, Valley Forge, PA, 19482-0980, US

(21) Appl. No.: 09/828,538

(22) Filed: Apr. 6, 2001

Related U.S. Application Data
(63) Continuation of application No. 09/328,014, filed on Jun. 8, 1999.
(60) Provisional application No. 60/088,524, filed on Jun. 8, 1998.
(60) Provisional application No. 60/093,726, filed on Jul. 22, 1998.

(51) Int. Cl. A61K 48/00; C12Q 1/68; C07H 21/04; C12N 5/06; C12P 21/02; C07K 14/705

(52) U.S. Cl. 514/44; 435/6; 435/69.1; 435/325; 530/350; 536/23.5

(57) ABSTRACT

The invention relates to HFGAN72 receptor polypeptides, polynucleotides encoding the polypeptides, methods for producing the polypeptides, in particular by expressing the polynucleotides, and agonists and antagonists of the polypeptides. The invention further relates to methods for utilizing such polynucleotides and polypeptides, as well as variants thereof, for applications, which relate, in part, to research, diagnostic and clinical arts.

### CROSS-REFERENCE TO RELATED APPLICATION

This application is a continuation of U.S. patent application No. 09/328,014, filed Jun. 8, 1999, (status: pending) which claims benefit to the earlier provisional U.S. Application Nos. 60/088,524, filed on Jun. 8, 1998, and 60/093,726, filed on Jul. 22, 1998, the contents of which are incorporated herein by reference in their entirety.

### FIELD OF THE INVENTION

This invention relates, in part, to newly identified polynucleotides and polypeptides; variants and derivatives of the polynucleotides and polypeptides; processes for making the polynucleotides and the polypeptides, and their variants and derivatives; agonists and antagonists of the polypeptides; and uses of the polynucleotides, polypeptides, variants, derivatives, agonists and antagonists. In particular, in these and in other regards, the invention relates to polynucleotides and polypeptides of HFGAN72X and HFGAN72Y receptors (hereinafter collectively referred to as HFGAN72 receptors), especially genomic sequences of HFGAN72 receptors, and most especially, promoter and intronic sequences.

### BACKGROUND OF THE INVENTION

It is well established that many medically significant biological processes are mediated by proteins participating in signal transduction pathways that involve G-proteins and/or second messengers, e.g., cAMP (Lefkowitz, Nature, 1991, 351:353-354). Herein, these proteins are referred to as proteins participating in pathways with G-proteins or PPG proteins. Some examples of these proteins include the GPC receptors, such as those for adrenergic agents and dopamine (Kobilka, B. K., et al., Proc. Natl Acad. Sci., USA, 1987, 84:46-50; Kobilka, B. K., et al., Science, 1987, 238:650-656; Bunzow, J. R, et al., Nature, 1988, 336:783-787), G-proteins themselves, effector proteins, e.g., phospholipase C, adenyl cyclase, and phosphodiesterase, and actuator proteins, e.g., protein kinase A and protein kinase C (Simon, M. I., et al., Science, 1991, 252:802-8).

For example, in one form of signal transduction, the effect of hormone binding is the activation of the enzyme, adenylate cyclase, inside the cell. Enzyme activation by hormones is dependent on the presence of the nucleotide GTP. GTP also influences hormone binding. A G-protein connects the hormone receptor to adenylate cyclase. G-protein was shown to exchange GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors (otherwise known as 7TM receptors) have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or famesylation) of cysteine residues can influence signal transduction of some G-protein coupled receptors. Most G-protein coupled receptors contain potential phosphorylation sites win the third cytoplasmic loop and/or the carboxy terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise hydrophilic sockets formed by several G-protein coupled receptor transmembrane domains, said socket being surrounded by hydrophobic residues of the G-protein coupled receptors. The hydrophilic side of each G-protein coupled receptor transmembrane helix is postulated to face inward and form polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331) Different G-protein α-subunits preferentially stimulate particular effectors to modulate various biological functions in a cell. Phosphorylation of cytoplasmic residues of G-protein coupled receptors has been identified as an important mechanism for the regulation of G-protein coupling of some G-protein coupled receptors. G-protein coupled receptors are found in numerous sites within a mammalian host.

Over the past 15 years, hundreds of therapeutic agents targeting 7 transmembrane (7 TM) receptors have been successfully introduced onto the market.

This indicates that these receptors have an established, proven history as therapeutic targets. Clearly, there is a need for identification and characterization of further receptors which can play a role in preventing, ameliorating or correcting dysfunctions or diseases, including, but not limited to, infections such as bacterial, fungal, protozoan and viral infections, particularly infections caused by HIV-1 or HIV-2; cancers; diabetes; asthma; Parkinson's disease; both acute and congestive heart failure; hypotension; hypertension; urinary retention; osteoporosis; angina pectoris; myocardial infarction; ulcers; asthma; allergies; benign prostatic hypertrophy; chronic renal failure; renal disease; impaired glucose tolerance; seizure disorder; depression; anxiety; obsessive compulsive disorder; affective neurosis/disorder; depressive neurosis/disorder; anxiety neurosis; dysthymic disorder; behavior disorder; mood disorder; schizophrenia; psychosexual dysfunction; sex disorder; sexual disorder; disturbed biological and circadian rhythms; feeding disorders, such as anorexia, bulimia, cachexia, and obesity; Cushing's syndrome/disease; basophil adenoma; prolactinoma; hyperprolactinemia; hypopituitarism; hypophysis tumor/adenoma; hypothalamic diseases; Froehlich's syndrome; adenohypophysis disease; hypophysis disease; hypophysis tumor/adenoma; pituitary growth hormone; adenohypophysis hypofunction; adrenohpophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, heart and lung diseases, mental illness, and addictions; migraine; hyperalgesia; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labour pain; cancer pain; post-chemotherapy pain; post-stroke pain; post-operative pain; neuralgia; and tolerance to narcotics or withdrawal from narcotics; sleep disorders; sleep apnea; narcolepsy; insomnia; parasomnia; jet-lag syndrome; and other neurodegenerative disorders, which includes nosological entities such as disinhibition-dementia-parkinsonism-amyotrophy complex; pallido-ponto-nigral degeneration; and dyskinesias, such as Huntington's disease or Gilles dela Tourett's syndrome, among others.

### SUMMARY OF THE INVENTION

Toward these ends, and others, it is an object of the present invention to provide polypeptides, inter alia, that have been identified as novel HFGAN72 receptors.

It is a further object of the invention, moreover, to provide polynucleotides that encode HFGAN72 receptors, particularly polynucleotides that encode the polypeptides herein designated HFGAN72 receptors.

In a particularly preferred embodiment of this aspect of the invention, the polynucleotide comprises the region encoding HFGAN72 receptors in the sequences set forth in SEQ ID NOs:1-17.

In a particularly preferred embodiment of this aspect of the invention the polynucleotide comprises the region encoding human HFGAN72 receptors in the sequence set out in SEQ ID NOs:1-17 or in the genomic DNA (herein “gDNA”) in ATCC Deposit No. 98806 (referred to herein as the deposited clone).

In accordance with this aspect of the invention there are provided isolated nucleic acid molecules encoding HFGAN72 receptors, including mRNAs, cDNAs, genomic DNAs and, in further embodiments of this aspect of the invention, biologically, diagnostically, clinically or therapeutically useful variants, analogs or derivatives thereof, or fragments thereof, including fragments of the variants, analogs and derivatives, such as the variant HFGAN72 receptor gDNA set forth in FIG. 4 [SEQID NOs:21 and 22].

Among the particularly preferred embodiments of this aspect of the invention are naturally occurring allelic variants of HFGAN72 receptors.

It also is an object of the invention to provide HFGAN72 receptor polypeptides, such as HFGAN72X set forth in FIG. 2 [SEQ ID NO:19] and HFGAN72Y [SEQ ID NO:20] set forth in FIG. 3 particularly human HFGAN72 receptor polypeptides, that cause or are associated with disease, for example infections such as bacterial, fill protozoan and viral infections, particularly infections caused by HIV-1 or HIV-2; cancers; diabetes; asthma; Parkinson's disease; both acute and congestive heart failure; hypotension; hypertension; urinary retention; osteoporosis; angina pectoris; myocardial infarction; ulcers; asthma; allergies; benign prostatic hypertrophy; chronic renal failure; renal disease; impaired glucose tolerance; seizure disorder; depression; anxiety; obsessive compulsive disorder; affective neurosis/disorder; depressive neurosis/disorder; anxiety neurosis; dysthymic disorder; behavior disorder; mood disorder; schizophrenia; psychosexual dysfunction; sex disorder; sexual disorder; disturbed biological and circadian rhythms; feeding disorders, such as anorexia, bulimia, cachexia, and obesity; Cushing's syndrome/disease; basophil adenoma; prolactinoma; hyperprolactinemia; hypopituitarism; hypophysis tumor/adenoma; hypothalamic diseases; Froelich's syndrome; adenohypophysis disease; hypophysis disease; hypophysis tumor/adenoma, pituitary growth hormone; adenohypophysis hypofunction; adrenohpophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, heart and lung diseases, mental illness, and addictions; migraine; hyperalgesia; enhanced or exaggerated sensitivity to pain, such as hyperalgesia, causalgia and allodynia; acute pain; bum pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labour pain; cancer pain; post-chemotherapy pain; post-stroke pain; post-operative pain; neuralgia; and tolerance to narcotics or withdrawal from narcotics; sleep disorders; sleep apnea; narcolepsy; insomnia; parasomnia; jet-lag syndrome; and other neurodegenerative disorders, which includes nosological entities such as disinhibition-dementia-parkinsonism-amyotrophy complex; pallido-ponto-nigral degeneration; and dyskinesias, such as Huntington's disease or Gilles dela Tourett's syndrome, among others.

In accordance with this aspect of the invention there are provided novel polypeptides of human origin referred to herein as HFGAN72 receptors as well as biologically, diagnostically or therapeutically useful fragments, variants and derivatives thereof, variants and derivatives of the fragments, and analogs of the foregoing.

Among the particularly preferred embodiments of this aspect of the invention are variants of human HFGAN72 receptors encoded by naturally occurring alleles of the human HFGAN72 receptor gene, such as the variant genomic polynucleotide sequence set forth in FIG. 4 [SEQ ID NOs:21-22] and the encoded polypeptide sequence set forth in FIG. 6 [SEQ ID NO:24], among others.

It is another object of the invention to provide a process for producing the aforementioned polypeptides, polypeptide fragments, variants and derivatives, fragments of the variants and derivatives, and analogs of the foregoing.

In a preferred embodiment of this aspect of the invention there are provided methods for producing the aforementioned HFGAN72 receptor polypeptides comprising culturing host cells having expressibly incorporated therein an exogenously-derived human HFGAN72 receptor-encoding polynucleotide under conditions for expression of human HFGAN72 receptor in the host and then recovering the expressed polypeptide.

In accordance with yet another object of the invention there are methods to determine drug responsiveness of individuals having or suspected of having a defect in the HFGAN72 receptor gene.

In accordance with yet another object the invention there are provided products, compositions, processes and methods that utilize the aforementioned polypeptides and polynucleotides for research, biological, clinical and therapeutic purposes, inter alia.

In accordance with certain preferred embodiments of this aspect of the invention, there are provided products, compositions and methods, inter alia, for, among other things: assessing HFGAN72 receptor expression in cells by determining HFGAN72 receptor polypeptides of HFGAN72 receptor-encoding mRNA or hnRNA in vitro, ex vivo or in vivo by exposing cells to HFGAN72 receptor polypeptides, polynucleotides or antibodies as disclosed herein; assaying genetic variation and aberrations, such as defects, in HFGAN72 receptor polynucleotides, genes and gene control sequences; and administering a HFGAN72 receptor polypeptide or polynucleotide to an organism to augment HFGAN72 receptor function or remediate HFGAN72 receptor dysfunction.

In accordance with certain preferred embodiments of this and other aspects of the invention there are provided probes that hybridize specifically to human HFGAN72 receptor sequences.

In certain additional preferred embodiments of this aspect of the invention, there are provided antibodies against HFGAN72 receptor polypeptides. In certain particularly preferred embodiments in this regard, the antibodies are highly selective for human HFGAN72 receptor.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

The following drawings depict certain embodiments of the invention. They are illustrative only and do not limit the invention otherwise disclosed herein.

FIG. 1 [SEQ ID NOs:1-18] shows part of the genomic nucleotide sequence of the HFGAN72 receptors. Exons are capitalized and bolded. Intron sequence is in lower case letters. The areas in upper case are nucleotide residues contained in the genomic clone but not classified. These nucleotide residues could be additional 5′ and/or 3′ UTR for HFGAN72 X, HFGAN72Y, and/or the nucleotide sequence for other splice variants, or intronic sequence, etc. SEQ ID NO:1 (nucleotide residues 1-663) consists of the 5′ upstream sequence. SEQ ID NO:2 (nucleotide residues 664-1019 consists of exon 1. SEQ ID NO:3 (nucleotide residues 1020-1159) consists of intron 1. SEQ ID NO:4 (nucleotide residues 1160-1338) consists of exon 2. SEQ ID NO:5 (nucleotide residues 1339-2471) consists of intron 2. SEQ ID NO:6 (nucleotide residues 2472-2715) consists of exon 3. SEQ ID NO:7 (nucleotide residues 2716-3105) consists of intron 3. SEQ ID NO:8 (nucleotide residues 3106-3221) consists of exon 4. SEQ ID NO:9 (nucleotide residues 3222-5150) consists of intron 4. SEQ ID NO:10 (nucleotide residues 5151-5377) consists of exon 5. SEQ ID NO:11 (nucleotide residues 5378-6625) consists of intron 5. SEQ ID NO:12 (nucleotide residues 6626-6747) consists of exon 6. SEQ ID NO:13 (nucleotide residues 6748-8419) consists of intron 6. SEQ ID NO:14 (nucleotide residues 8420-8743) consists of exon 7. SEQ ID NO:15 (nucleotide residues 8744-10453) and SEQ ID NO:16 (nucleotide residues 12953-14217) consists of intron 7. SEQ ID NO:17 (nucleotide residues 14218-14313) consists of exon 8. SEQ ID NO:18 (nucleotide residues 14314-14780) consists of the 3′ downstream sequence.

FIG. 2 [SEQ ID NO:19] shows the deduced amino acid sequence of the HFGAN72X receptor.

FIG. 3 [SEQ ID NO:20] shows the deduced amino acid sequence of the HFGAN72Y receptor.

FIG. 4 [SEQ ID NOs:21-22] shows part of the genomic nucleotide sequence of a variant of the HFGAN72 receptors. Exons are capitalized and bolded. Intron sequence is in lower case letters. The areas in upper case are nucleotide residues contained in the genomic clone but not classified. These nucleotide residues could be additional 5′ and/or 3′ UTR for HFGAN72 X, HFGAN72Y, and/or the nucleotide sequence for other splice variants, or intronic sequence, etc. Polymorphisms in the exons are bolded and underlined, while the polymorphisms in the intronic sequence are bolded only.

FIG. 5 [SEQ ID NO:23] shows the polynucleotide sequence of the HFGAN72X variant encoded by the variant genomic polynucleotide sequence of the HFGAN72 receptors set forth in FIG. 4 [SEQ ID NOs:21-22]. The SNPs at residues 264 and 1375 are shown in bold. The SNP at residue 264 causes a silent change from arginine to argine, while the SNP at residue 1375 causes an amino acid change from isoleucine to valine.

FIG. 6 [SEQ ID NO:24] shows the deduced amino acid sequence of the HFGAN72X variant encoded by the variant genomic polynucleotide sequence of the HFGAN72 receptor set forth in FIG. 4 [SEQ ID NOs:21-22]. The single amino acid changed caused by the SNP at amino acid 408 is shown in bold.

### GLOSSARY

The following illustrative explanations are provided to facilitate understanding of certain terms used frequently herein, particularly in the examples. The explanations are provided as a convenience and are not limitative of the invention.

DIGESTION of DNA refers to catalytic cleavage of the DNA with a restriction enzyme that acts only at certain sequences in the DNA. The various restriction enzymes referred to herein are commercially available and their reaction conditions, cofactors and other requirements for use are known and routine to the skilled artisan.

For analytical purposes, typically, 1 μg of plasmid or DNA fragment is digested with about 2 units of enzyme in about 20 ml of reaction buffer. For the purpose of isolating DNA fragments for plasmid construction, typically 5 to 50 μg of DNA are digested with 20 to 250 units of enzyme in proportionately larger volumes.

Appropriate buffers and substrate amounts for particular restriction enzymes are described in standard laboratory manuals, such as those referenced below, and they are specified by commercial suppliers.

Incubation times of about 1 hour at 37° C. are ordinarily used, but conditions may vary in accordance with standard procedures, the supplier's instructions and the particulars of the reaction. After digestion, reactions may be analyzed, and fragments may be purified by electrophoresis through an agarose or polyacrylamide gel, using well known methods that are routine for those skilled in the art.

GENETIC ELEMENT generally means a polynucleotide comprising a region that encodes a polypeptide or a region that regulates transcription or translation or other processes important to expression of the polypeptide in a host cell, or a polynucleotide comprising both a region that encodes a polypeptide and a region operably linked thereto that regulates expression.

Genetic elements may be comprised within a vector that replicates as an episomal element; that is, as a molecule physically independent of the host cell genome. They may be comprised within mini-chromosomes, such as those that arise during amplification of transfected DNA by methotrexate selection in eukaryotic cells. Genetic elements also may be comprised within a host cell genome; not in their natural state but, rather, following manipulation such as isolation, cloning and introduction into a host cell in the form of purified DNA or in a vector, among others.

IDENTITY is a measure of the identity of nucleotide sequences or amino acid sequences. In general, the sequences are aligned so that the highest order match is obtained. “Identity” per se has an art-recognized meaning and can be calculated using published techniques (see, e.g.: COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCG program package (Devereux, et al., Nucleic Acids Research (1984) 12(1):387), BLASTP, BLASTN, and FASTA (Atschul, et al., J Molec Biol (1990) 215:403).

By way of example, a polynucleotide sequence of the present invention may be identical to the reference sequence of SEQ ID NO:2, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:2 by the numerical percent of the respective percent identity(divided by 100) and subtracting that product from said total number of nucleotides in SEQ ID NO:2, or:

nₙ≦xₙ−(xₙ·y),

wherein nₙ is the number of nucleotide alterations, xₙ is the total number of nucleotides in SEQ ID NO:2, and y is 0.80 for 80%, 0.85 for 85%, 0.90 for 90%, 0.95 for 95%, 0.97 for 97% or 1.00 for 100%, and wherein any non-integer product of xₙ and y is rounded down to the nearest integer prior to subtracting it from xₙ. Alterations of the polynucleotide sequence of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence encoded by SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO:2 by the numerical percent of the respective percent identity(divided by 100) and then subtracting that product from said total number of amino acids in the polypeptide sequence encoded by SEQ ID NO:2, or:

nₐ≦xₐ−(xₐ·y),

wherein nₐ is the number of amino acid alterations, x_{a,} is the total number of amino acids in the polypeptide sequence encoded by SEQ ID NO:2, and y is, for instance 0.80 for 80%, 0.85 for 85% etc., and wherein any non-integer product of x_{a,} and y is rounded down to the nearest integer prior to subtracting it from xₐ.

“Fusion protein” refers to a protein encoded by two, often unrelated, fused genes or fragments thereof In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

ISOLATED means altered “by the hand of man” from its natural state; i.e., that, if it occurs in nature, it has been changed or removed from its original environment, or both.

For example, a naturally occurring polynucleotide or a polypeptide naturally present in a living animal in its natural state is not “isolated,” but the same polynucleotide or polypeptide separated from some or all of the coexisting materials of its natural is “isolated”, as the term is employed herein.

As part of or following isolation, such polynucleotides can be joined to other polynucleotides, such as DNAs, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the polynucleotides and polypeptides may occur in a composition, such as a media formulations, solutions for introduction of polynucleotides or polypeptides, for example, into cells, compositions or solutions for chemical or enzymatic reactions, for instance, which are not naturally occurring compositions, and, therein remain isolated polynucleotides or polypeptides within the meaning of that term as it is employed herein.

LIGATION refers to the process of forming phosphodiester bonds between two or more polynucleotides, which most often are double stranded DNAs. Techniques for ligation are well known to the art and protocols for ligation are described in standard laboratory manuals and references, such as, for instance, Sambrook et al., MOLECULAR CLONING, A LABORATORY MANUAL, 2nd Ed.; Cold Spring Harbor Laboratory Press, Cold Spring Harbor, N.Y. (1989) and Maniatis et al., pg. 146, as cited below.

OLIGONUCLEOTIDE(S) refers to relatively short polynucleotides. Often the term refers to single-stranded deoxyribonucleotides, but it can refer as well to single-, double-, or triple-stranded ribonucleotides, antisense polynucleotides, RNA:DNA hybrids and double-stranded DNAs, among others.

Oligonucleotides, such as single-stranded DNA probe oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms.

Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP.

The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well known, this reaction can be prevented selectively, where desired, by removing the 5′ phosphates of the other polynucleotide(s) prior to ligation.

PLASMIDS generally are designated herein by a lower case letter p preceded and/or followed by capital letters and/or numbers, in accordance with standard naming conventions that are familiar to those of skill in the art.

Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

POLYNUCLEOTIDE(S) generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single-and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

POLYPEPTIDES, as used herein, includes all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modifications that occur naturally in polypeptides are too numerous to list exhaustively here, but they are well described in basic texts and in more detailed monographs, as well as in a voluminous research literature, and they are well known to those of skill in the art.

Among the known modifications which may be present in polypeptides of the present are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance PROTEINS—STRUCTURE AND MOLECULAR PROPERTIES, 2nd Ed., T. E. Creighton, W. H. Freeman and Company, New York (1993). Many detailed reviews are available on this subject, such as, for example, those provided by Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Analysis for protein modifications and nonprotein cofactors, Meth. Enzymol. 182: 626-646 (1990) and Rattan, et al., Protein Synthesis: Posttranslational Modifications and Aging, Ann. N.Y. Acad. Sci. 663: 48-62 (1992).

It will be appreciated, as is well known and as noted above, that polypeptides are not always entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well.

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is well known, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to express efficiently mammalian proteins having native patterns of glycosylation, inter alia. Similar considerations apply to other modifications.

It will be appreciated that the same type of modification may be present in the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

VARIANT(S) of polynucleotides or polypeptides, as the term is used herein, are polynucleotides or polypeptides that differ from a reference polynucleotide or polypeptide, respectively. Variants in this sense are described below and elsewhere in the present disclosure in greater detail.

(1) A polynucleotide that differs in nucleotide sequence from another, reference polynucleotide. Generally, differences are limited so that the nucleotide sequences of the reference and the variant are closely similar overall and, in many regions, identical.

As noted below, changes in the nucleotide sequence of the variant may be silent. That is, they may not alter the amino acids encoded by the polynucleotide. Where alterations are limited to silent changes of this type a variant will encode a polypeptide with the same amino acid sequence as the reference. Also as noted below, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed below.

(2) A polypeptide that differs in amino acid sequence from another, reference polypeptide. Generally, differences are limited so that the sequences of the reference and the variant are closely similar overall and, in many region, identical.

A variant and reference polypeptide may differ in amino acid sequence by one or more substitutions, additions, deletions, fusions and truncations, which may be present in any combination.

### DESCRIPTION OF THE INVENTION

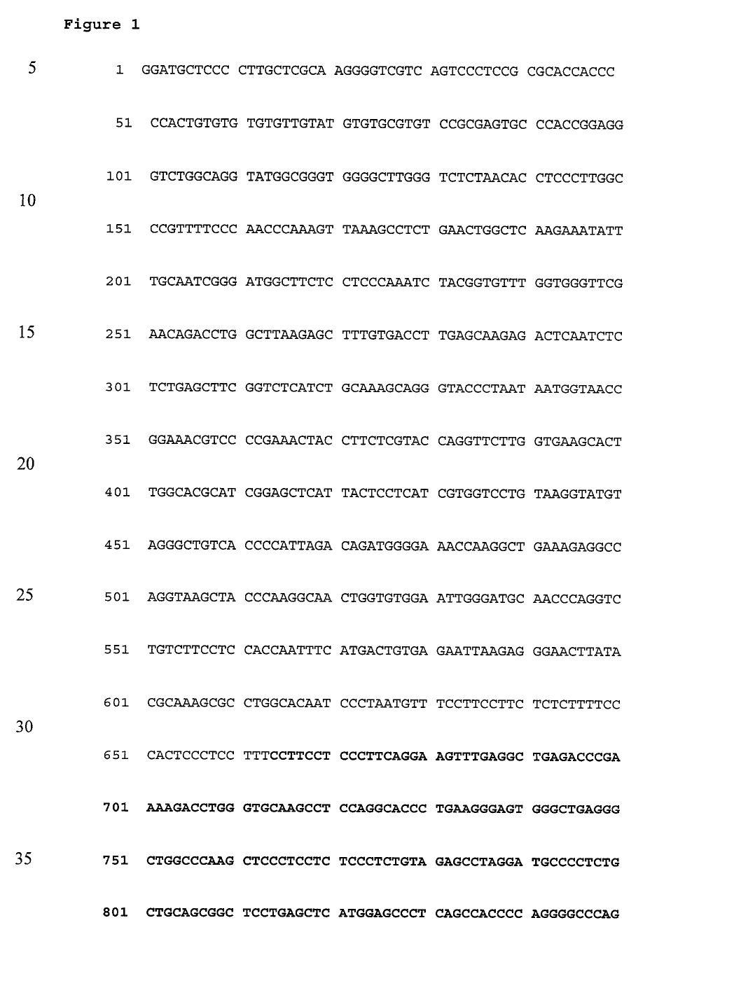

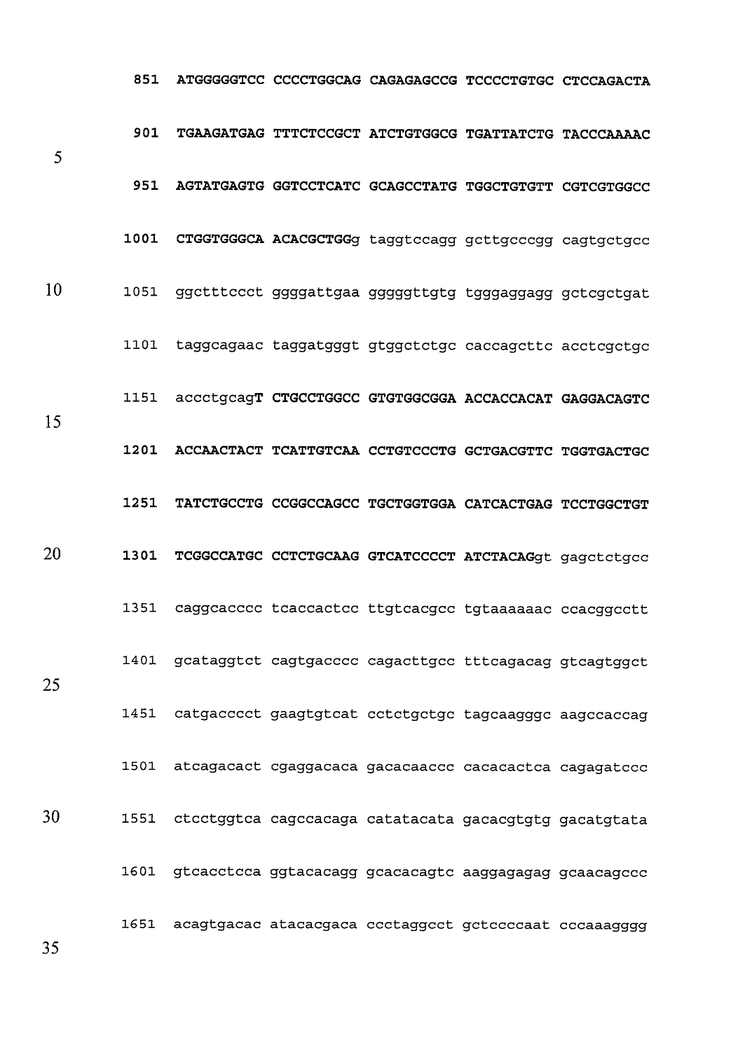

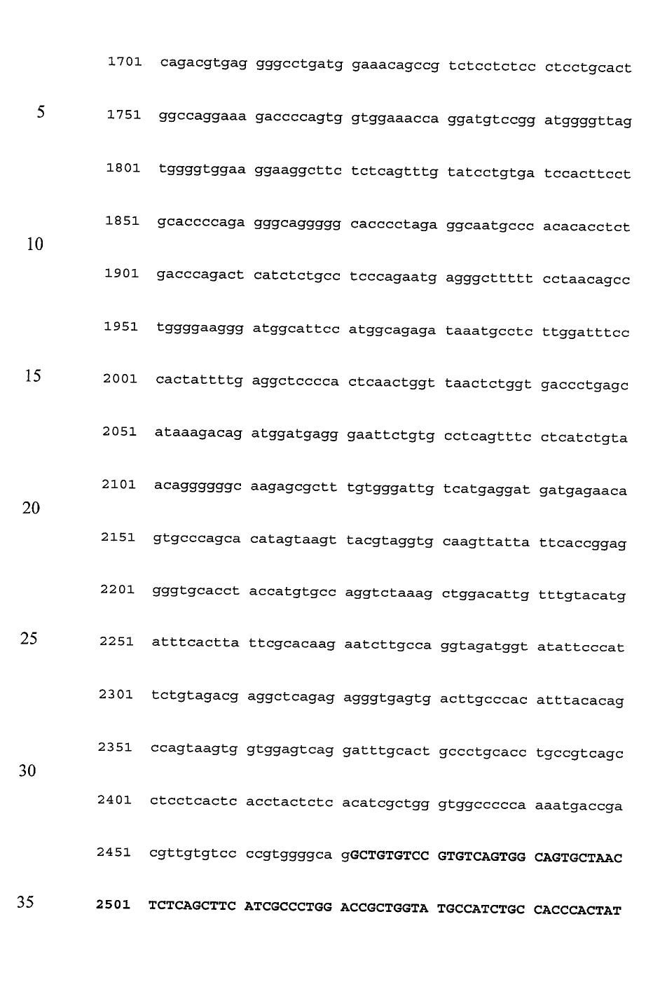

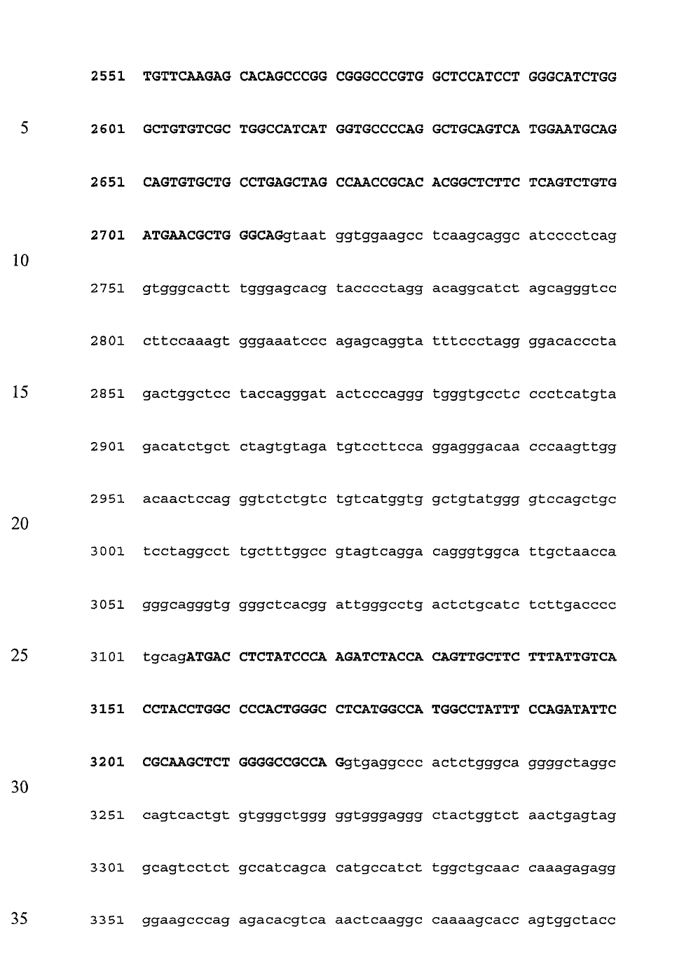

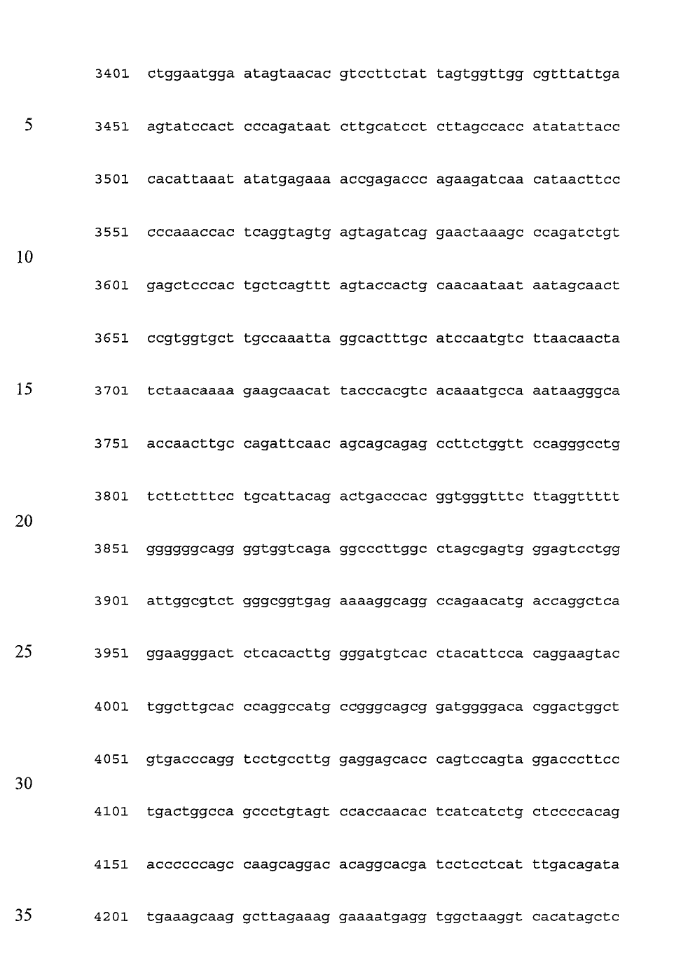

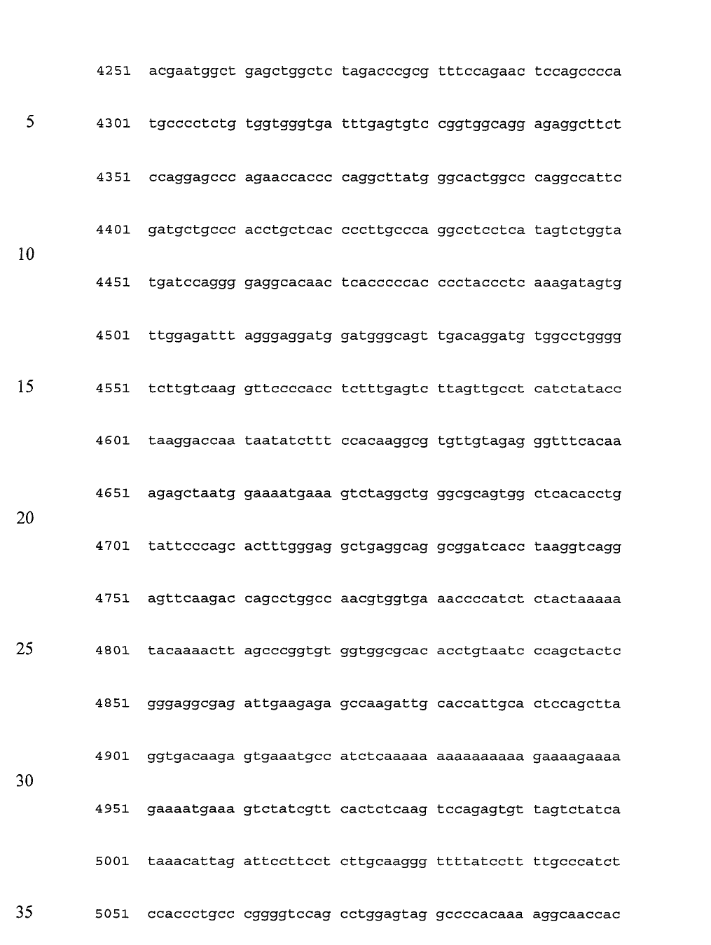

The present invention relates to novel HFGAN72 receptor polypeptides and polynucleotides, among other things, as described in greater detail below. The invention relates especially to human HFGAN72 receptors having the nucleotide sequences set out in FIG. 1 [SEQ ID NOs:1-18], and to the human HFGAN72 receptor nucleotide sequences of the gDNA in ATCC Deposit Number 98806, which is herein referred to as “the deposited clone” or as the “gDNA of the deposited clone.” It will be appreciated that the nucleotide sequences set out in FIG. 1 [SEQ ID NOs:1-18] were obtained by sequencing the gDNA of the deposited clone, as more specifically set forth elsewhere herein. Hence, the sequence of the deposited clone is controlling as to any discrepancies between the two.

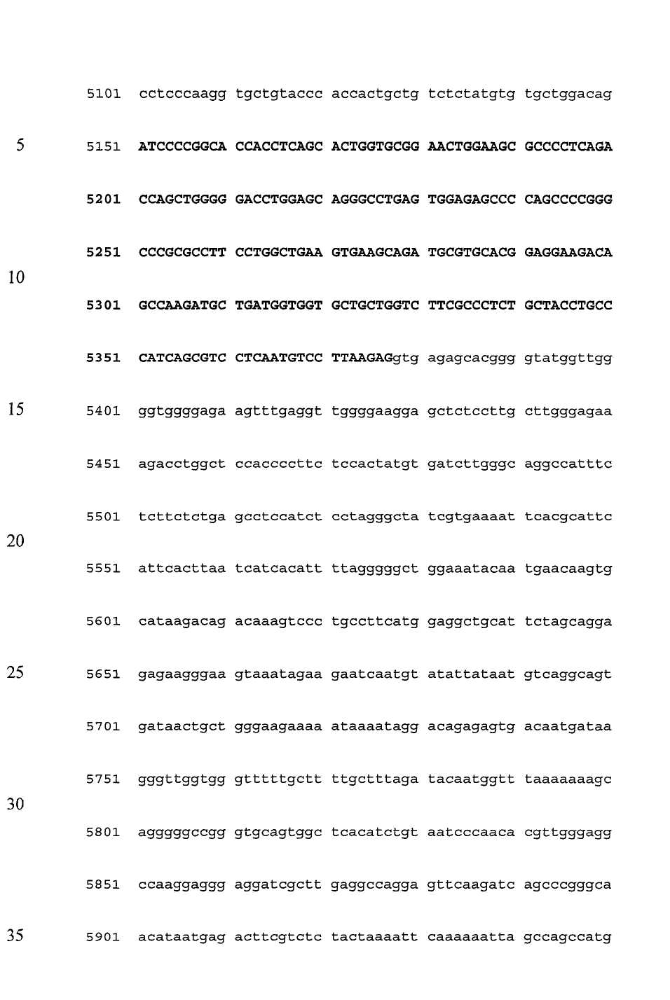

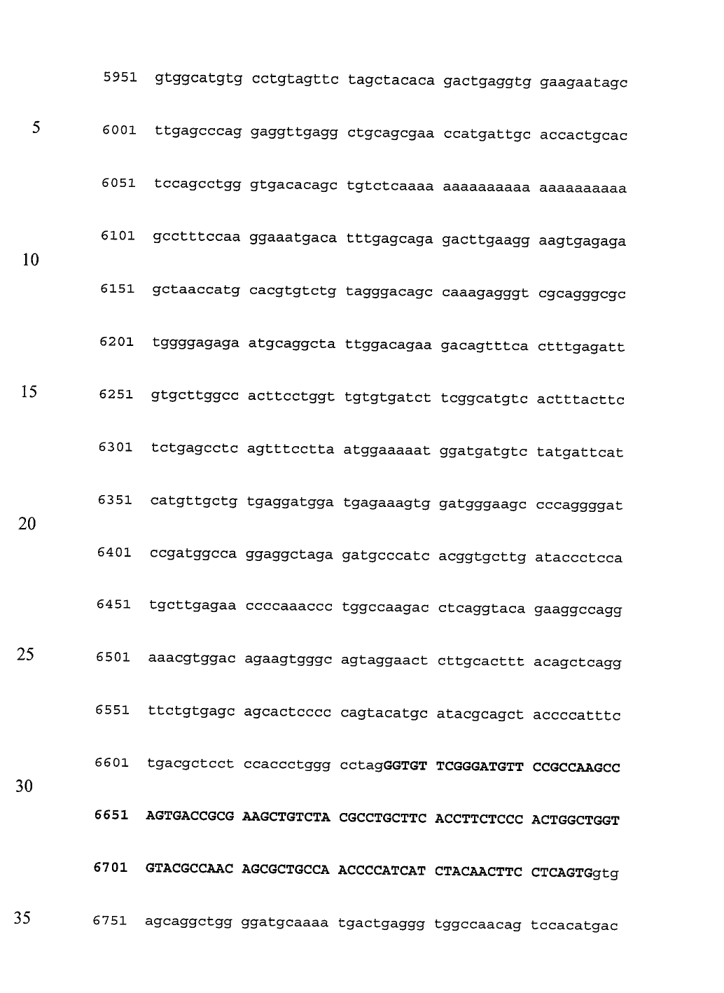

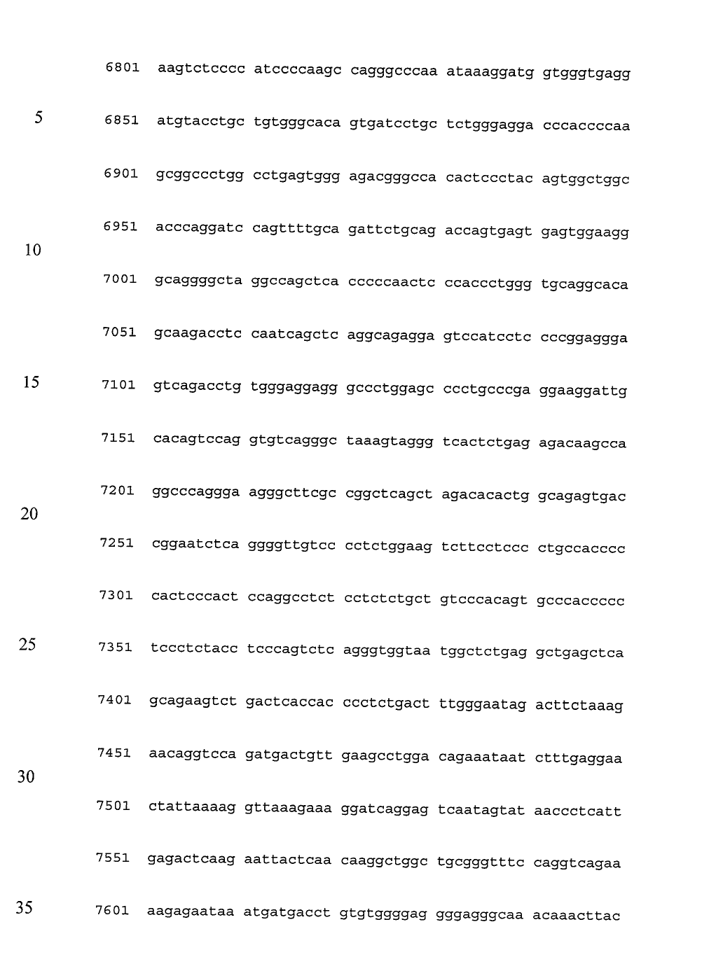

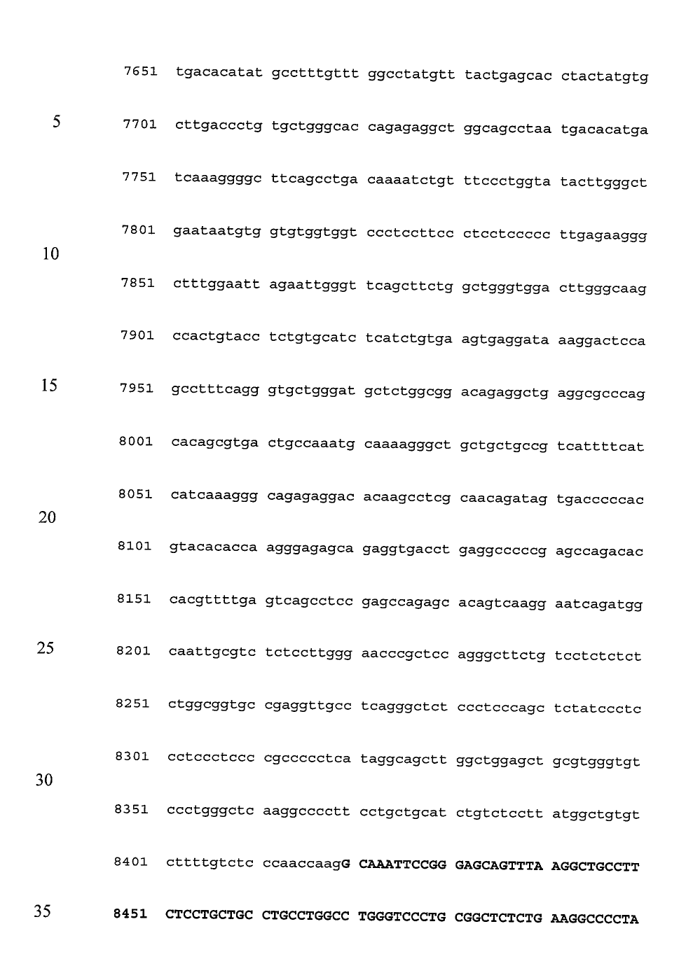

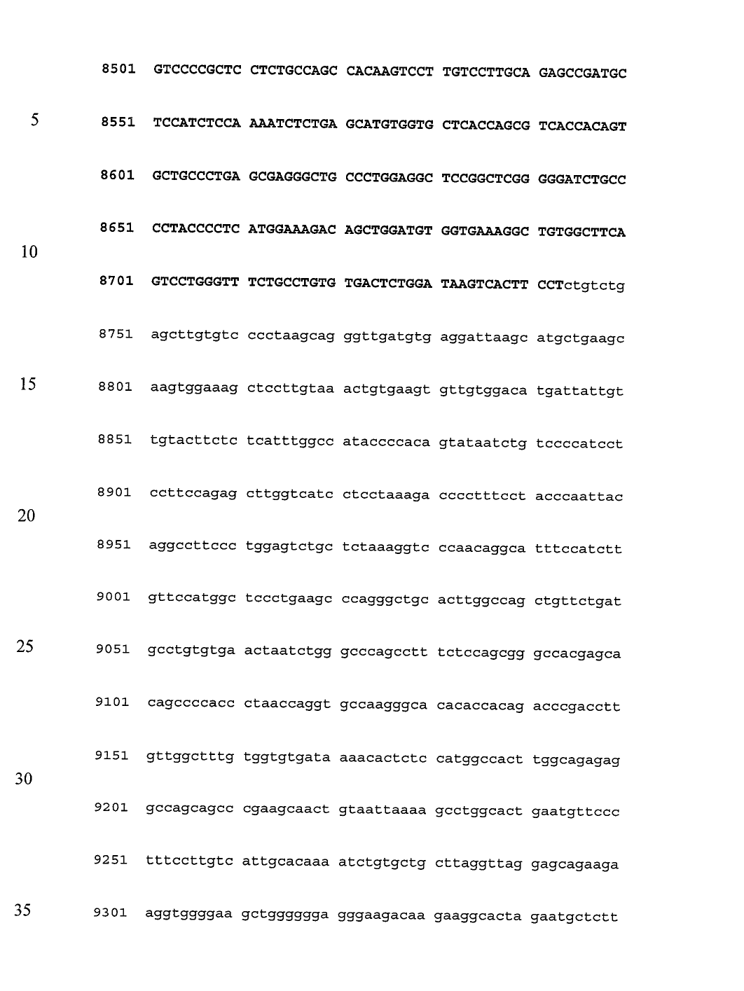

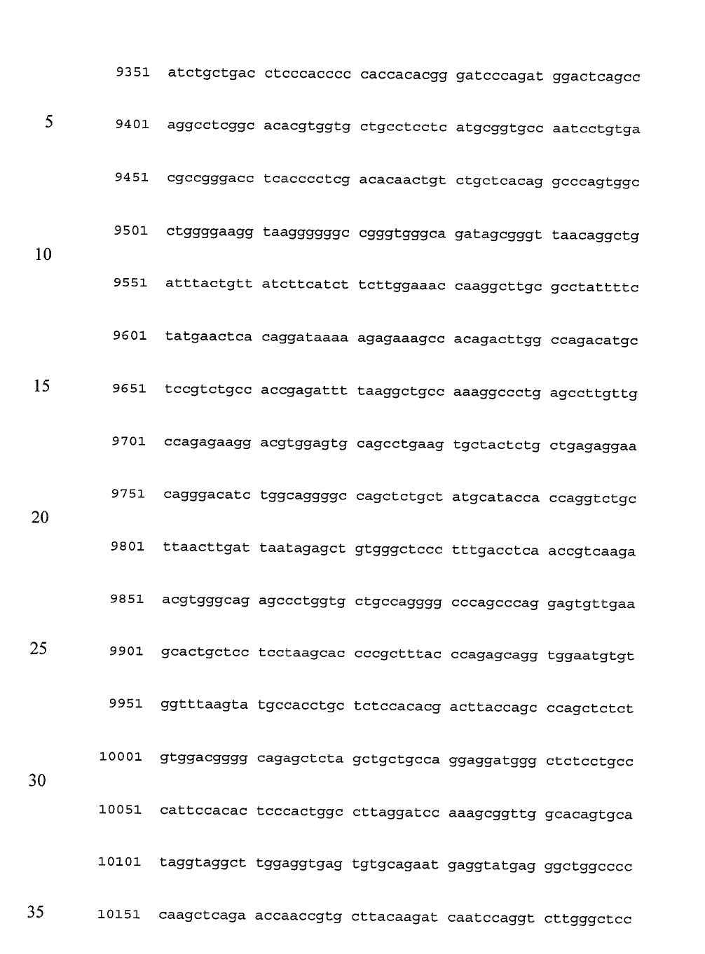

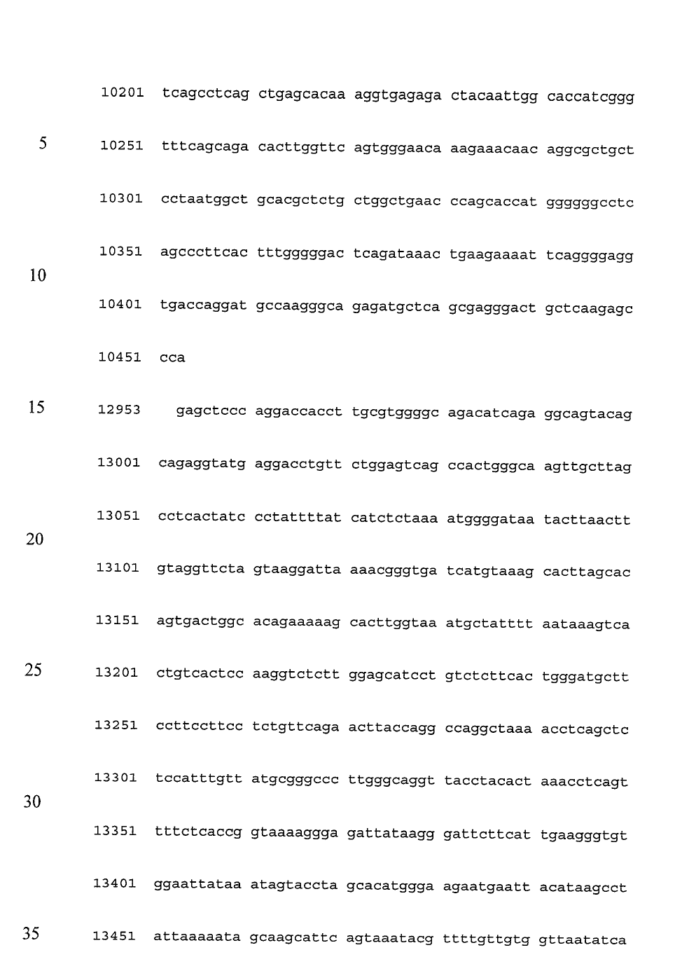

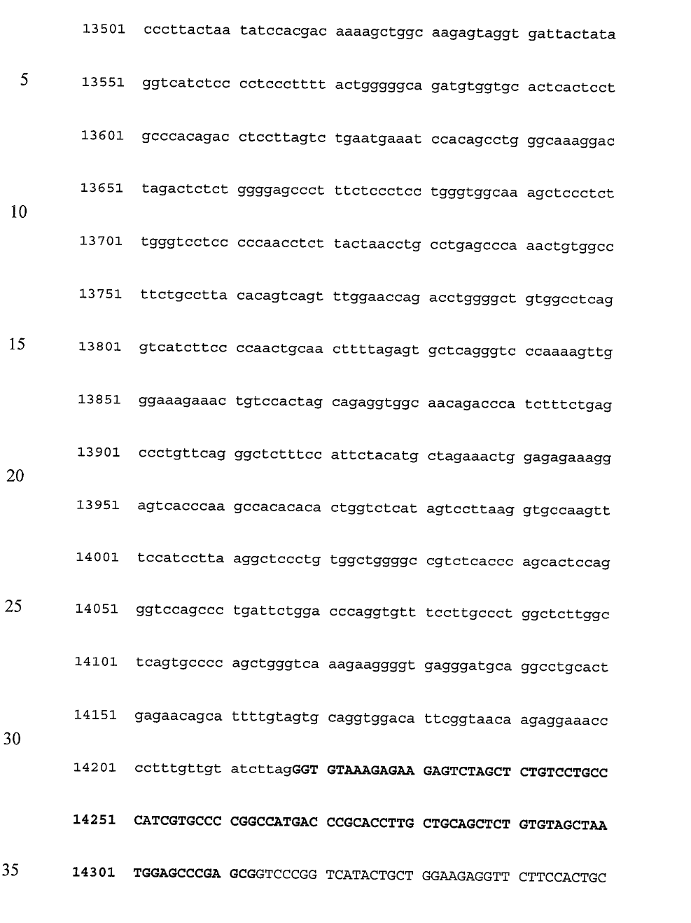

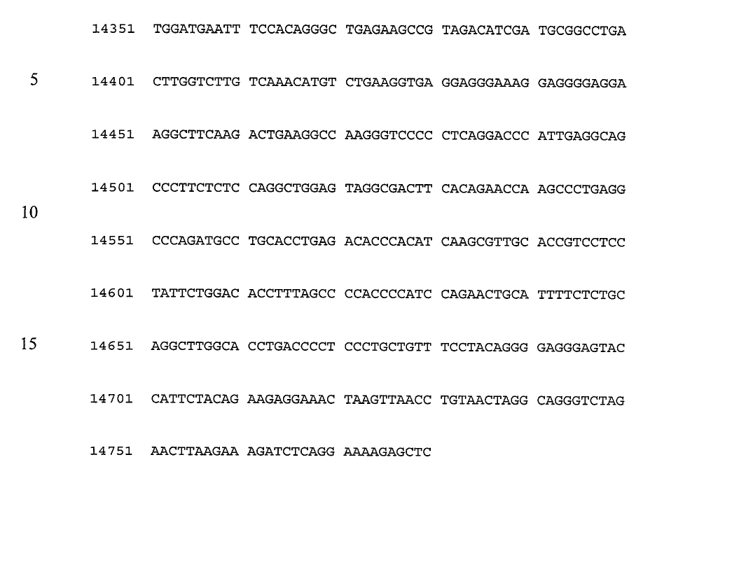

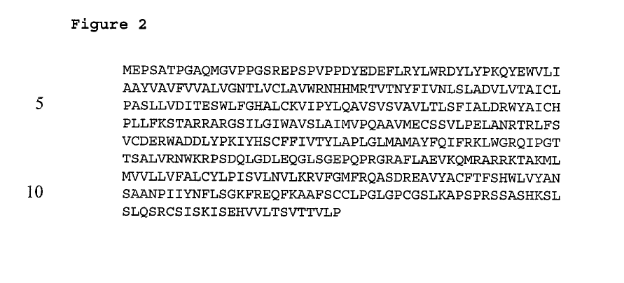

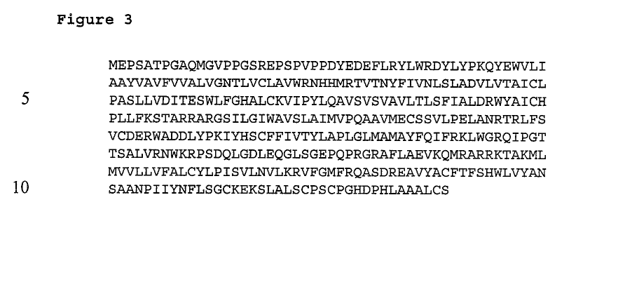

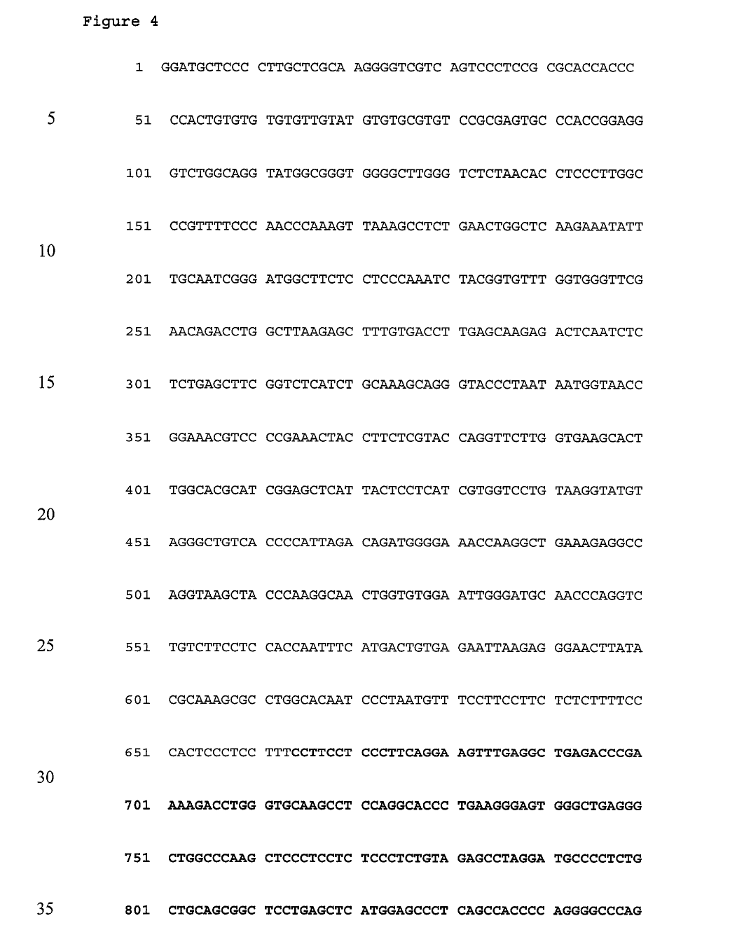

Furthermore, the present invention relates to the HFGAN72 receptor polypeptides encoded by the polynucleotides of FIG. 1 [SEQ ID NOs:1-18]. Specifically, the deduced amino acid sequence of the HFGAN72X receptor is set forth in FIG. 2 [SEQ ID NO:19], and the deduced amino acid sequence of the HFGAN72Y receptor is set forth in FIG. 3 [SEQ ID NO:20].

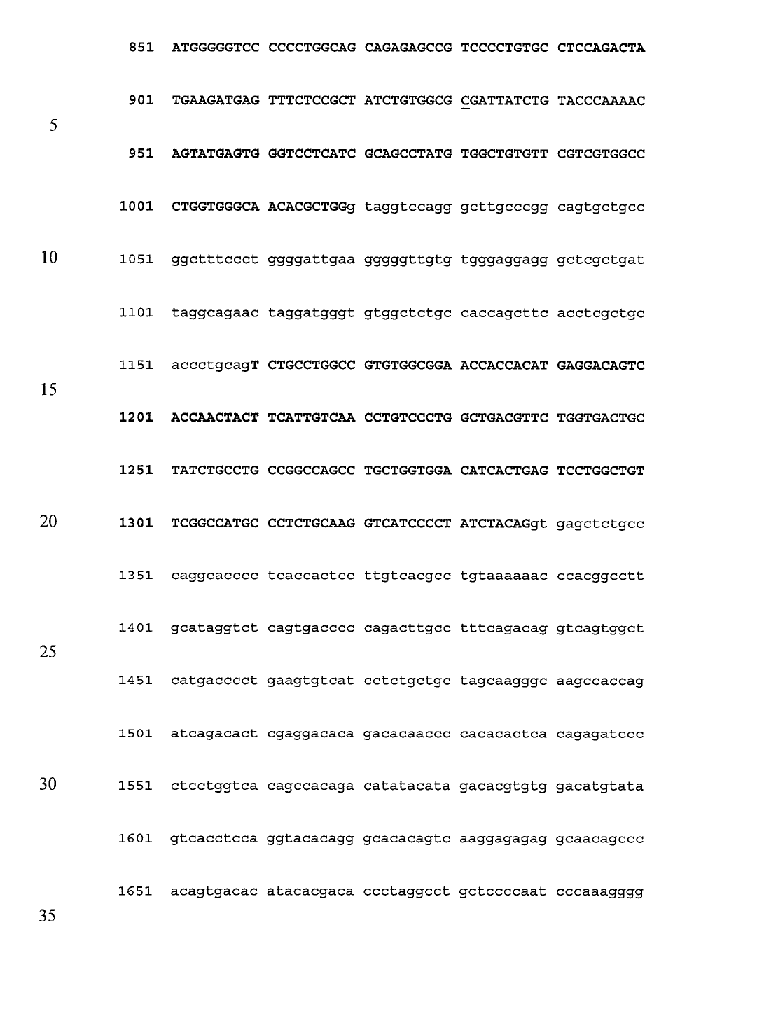

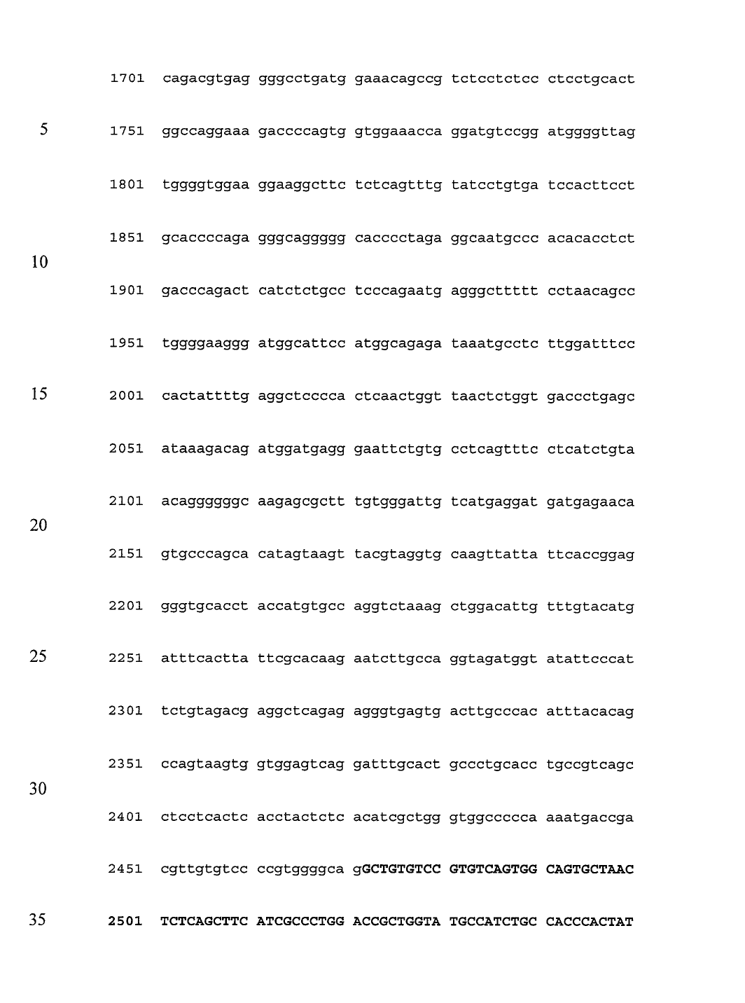

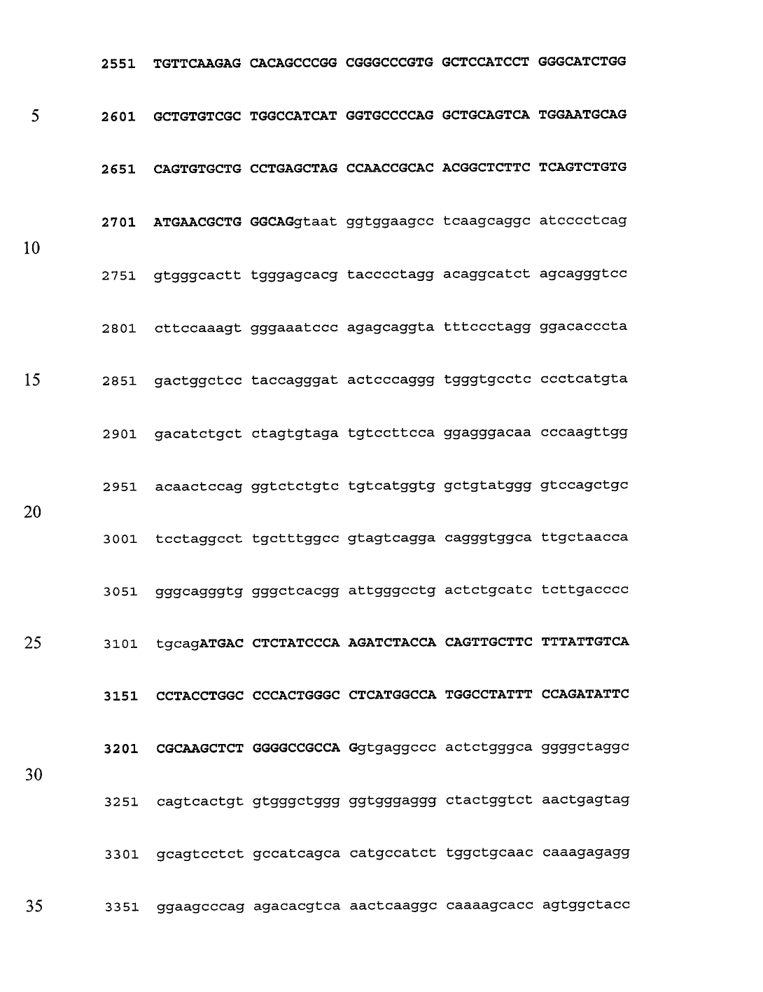

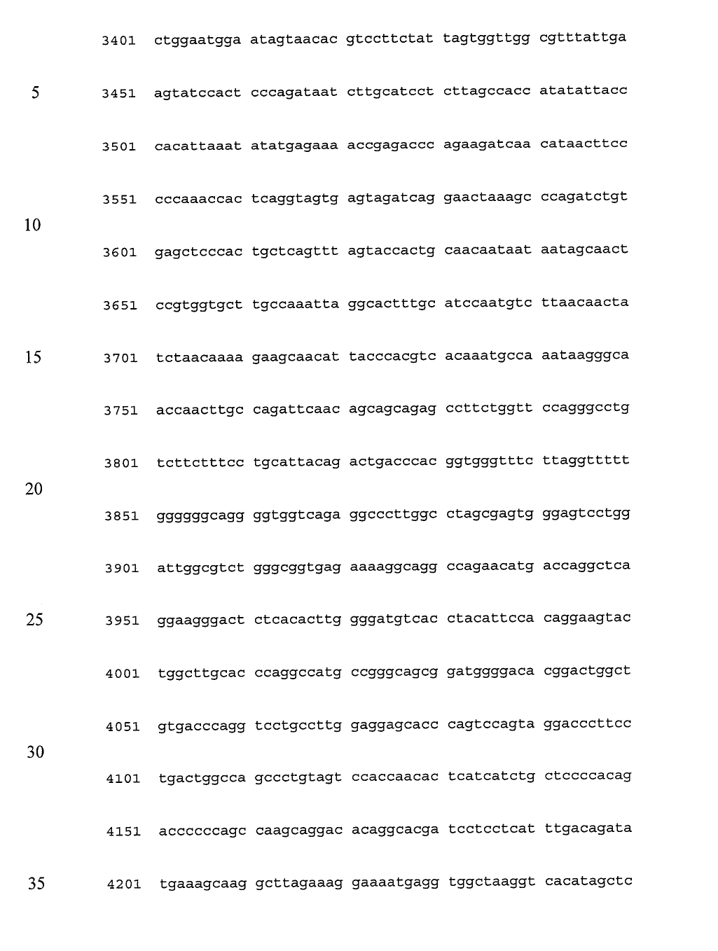

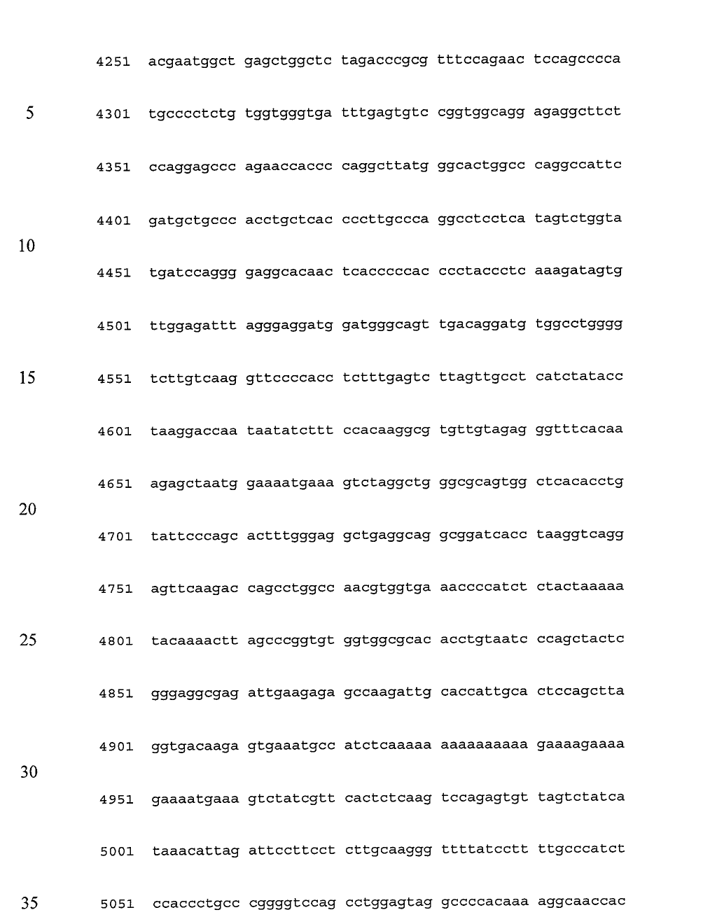

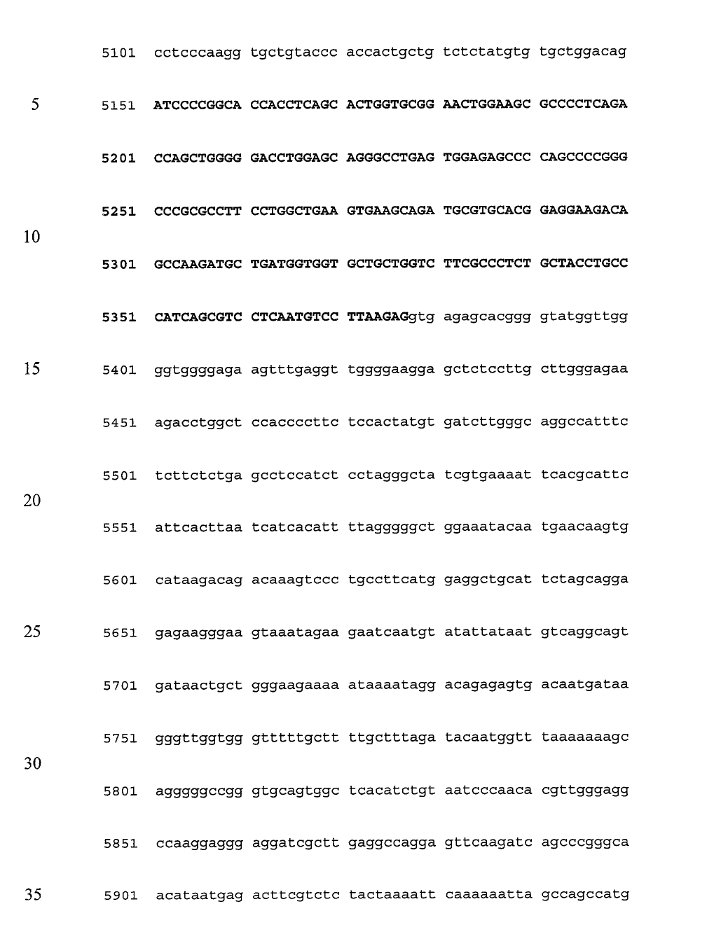

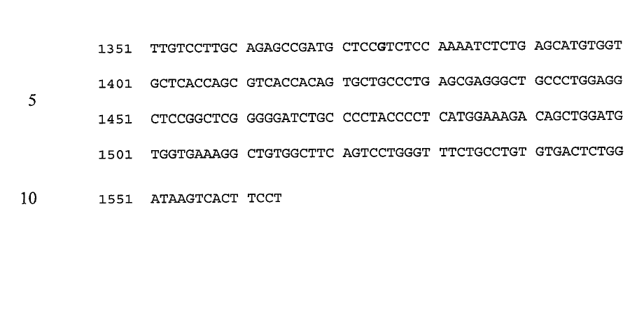

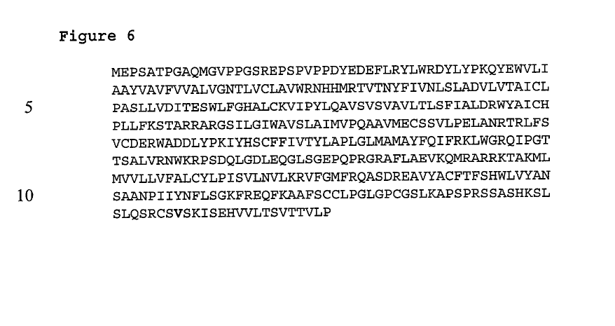

An alternative embodiment of this invention are variants of human HFGAN72 receptors encoded by naturally occurring alleles of the human HFGAN72 receptor gene, such as the variant genomic nucleotide sequence set forth in FIG. 4 [SEQ ID NOs:21-22], and the polypeptides encoded by this variant set forth in FIG. 6 [SEQ ID NO:24], among others.

Polynucleotides

In accordance with one aspect of the present invention, there are provided isolated polynucleotides which encode the HFGAN72 receptor polypeptides.

Using the information provided herein, such as the polynucleotide sequences set out in FIG. 1 [SEQ ID NOs:1-18], a polynucleotide of the present invention encoding human HFGAN72 receptor polypeptide may be obtained using standard cloning and screening procedures, such as those for cloning gDNAs using DNA from cells of a human as starting material. Illustrative of the invention, the polynucleotide set out in FIG. 1 [SEQ ID NOs:1-18] was discovered in a human gDNA library as described in Example 1.

Polynucleotides of the present invention may be in the form of RNA, such as mRNA or hnRNA, or in the form of DNA, including, for instance, cDNA and gDNA obtained by cloning or produced by chemical synthetic techniques or by a combination thereof The DNA may be triple-stranded, double-stranded or single-stranded. Single-stranded DNA may be the coding strand, also known as the sense strand, or it may be the non-coding strand, also referred to as the anti-sense strand.

The coding sequence which encodes the polypeptide may be identical to the exon sequence of the polynucleotides shown in FIG. 1 [SEQ ID NOs:2, 4, 6, 8, 10, 12, 14, and 17].

The polypeptides of the present invention, which are encoded by the polynucleotides set forth in FIG. 1 [SEQ ID NOs:2, 4, 6, 8, 10, 12, 14, and 17] may include, but are not limited to the coding sequence for the mature polypeptide, by itself, the coding sequence for the mature polypeptide and additional coding sequences, such as those encoding a leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing—including splicing and polyadenylation signals, for example—ribosome binding and stability of mRNA; additional coding sequence which codes for additional amino acids, such as those which provide additional functionalities. Thus, for instance, the polypeptide may be fused to a marker sequence, such as a peptide, which facilitates purification of the fused polypeptide. In certain preferred embodiments of this aspect of the invention, the marker sequence is a hexa-histidine peptide, such as the tag provided in the vector pQE-9, among others, many of which are commercially available. As described in Gentz, et al., Proc. Natl. Acad. Sci., USA 86: 821-824 (1989), for instance, hexa-histidine provides for convenient purification of the fusion protein. The HA tag corresponds to an epitope derived of influenza hemagglutinin protein, which has been described by Wilson, et al., Cell 37: 767 (1984), for instance.

In accordance with the foregoing, the term “polynucleotide encoding a polypeptide” as used herein encompasses polynucleotides which include a sequence encoding a polypeptide of the present invention. The term encompasses polynucleotides that include a single continuous region or discontinuous regions encoding the polypeptide (for example, interrupted by introns) together with additional regions, that also may contain coding and/or non-coding sequences.

The present invention further relates to variants of the herein above described polynucleotides which encode for fragments, analogs and derivatives of the polypeptide encoded by the exons of the gDNA set forth in FIG. 1 [SEQ ID NOs:2, 4, 6, 8, 10, 12, 14, and 17], including, but not limited to, splice variants transcribed from such gDNA. A variant of the polynucleotide may be a naturally occurring variant such as a naturally occurring allelic variant or splice variant, or it may be a variant that is not known to occur naturally. Such non-naturally occurring variants of the polynucleotide may be made by mutagenesis techniques, including those applied to polynucleotides, cells or organisms. Such non-naturally occurring variants of the polynucleotide may be made by modifying splice acceptor, donor and/or branch sites, or by expressing the gDNA in cells where it is not naturally expressed, or cell extracts made from such cells.

Among variants in this regard are variants that differ from the aforementioned polynucleotides by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions.

Among the particularly preferred embodiments of the invention in this regard is the variant polynucleotide sequence of HFGAN72 receptors set forth in the gDNA of FIG. 4 [SEQ ID NOs:21-22]; variants, analogs, derivatives and fragments thereof, and fragments of the variants, analogs and derivatives.

Further particularly preferred in this regard are polynucleotides encoding HFGAN72 receptor variants, analogs, derivatives and fragments, and variants, analogs and derivatives of the fragments, which comprise the HFGAN72X receptor polypeptide encoded by exons 1-7 of FIG. 1 [SEQ ID NOs:2, 4, 6, 8, 10, 12 and 14], and the HFGAN72Y receptor polypeptide encoded by exons 1-6 and 8 of FIG. 1 [SEQ ID NOs:2, 4, 6, 8, 10, 12, and 17] in which several, a few, 5 to 10, 1 to 5, 1 to 3, 2, 1 or no amino acid residues are substituted, deleted or added, in any combination. Especially preferred among these are silent substitutions, additions and deletions, which do not alter the properties and activities of the HFGAN72 receptors. Also especially preferred in this regard are conservative substitutions. Most highly preferred are polypeptides having the amino acid sequence set forth in FIG. 2 (SEQ ID NO:19), which comprises exons 1-7 (HFGAN72X), and the amino acid sequence set forth in FIG. 3 (SEQ ID NO:20), which comprises exons 1-6 and 8, (HFGAN72Y) of FIG. 1 [SEQ ID NOs:2, 4, 6, 8, 10, 12, 14, and 17] without substitutions.

Further preferred embodiments of the invention are polynucleotides that are at least 80% identical to a polynucleotide encoding either the HFGAN72X receptor polypeptide having the amino acid sequence set forth in FIG. 2 [SEQ ID NO:19] or the HFGAN72Y receptor polypeptide having the amino acid sequence set forth in FIG. 3 [SEQ ID NO:20], as well as variants, close homologs, derivatives and analogs thereof, as described above, and polynucleotides which are complementary to such polynucleotides. Alternatively, most highly preferred are polynucleotides that comprise a region that is at least 80% identical to a polynucleotide encoding the HFGAN72X and HFGAN72Y receptor polypeptides [SEQ ID NOs:2, 4, 6, 8, 10, 12, 14, and 17] and polynucleotides complementary thereto. In this regard, polynucleotides at least 95% identical to the same are particularly preferred. Furthermore, those with at least 97% are highly preferred among those with at least 95%, and among these those with at least 98% and at least 99% are particularly highly preferred, with at least 99% being the more preferred.

Still further preferred embodiments of the invention are polynucleotides comprising HFGAN72 receptor intron polynucleotide sequences, particularly polynucleotides comprising intron 1, 2, 3, 4, 5, 6 or 7, having the intron polynucleotide sequence set out in FIG. 1 [SEQ ID NOs:3, 5, 7, 9 ,11, 13, 15, and 16] or variants, close homologs, derivatives and analogs thereof, as described above, and polynucleotides which are complementary to such polynucleotides. Other preferred embodiments of the invention are polynucleotides comprising HFGAN72 receptor intron 1, 2, 3, 4, 5, 6 or 7 [SEQ ID NOs:3, 5, 7, 9, 11, 13, 15, and 16], operatively linked to the exon of a gene other than HFGAN72 receptor, or joining a HFGAN72 receptor exon and an exon of another gene.

Still other preferred embodiments of the invention are polynucleotides comprising HFGAN72 receptor exon polynucleotide sequences, particularly polynucleotides comprising exons 1-7 or 1-6 and 8, having the exon polynucleotide sequence set out in FIG. 1 [SEQ ID NOs:2, 4, 6, 8, 10, 12, 14, and 17] or variants, close homologs, derivatives and analogs thereof, as described above, and polynucleotides which are complementary to such polynucleotides. Other preferred embodiments of the invention are polynucleotides comprising HFGAN72 receptor exons 1-7 or 1-6 and 8, having the exon polynucleotide sequence set out in FIG. 1 [SEQ ID NOs:2, 4, 6, 8, 10, 12, 14, and 17], operatively linked to the intron of a gene other than HFGAN72 receptor.

Particularly preferred embodiments of the invention are differentially spliced polynucleotides which encode polypeptides which function in cells, especially those which have a biological activity of HFGAN72 receptors, most especially those expressed in human cells.

Polynucleotides comprising exon-exon pairs may be a naturally occurring variant such as a naturally occurring splice variant, or it may be a variant that is not known to occur naturally. Such non-naturally occurring variants of the polynucleotide may be made by mutagenesis techniques, including those applied to polynucleotides, cells or organisms. Such non-naturally occurring variants of the polynucleotide may be made by modifying splice acceptor, donor and/or branch sites, or by expressing the gDNA in cells where it is not naturally expressed, or cell extracts made from such cells. Exon-exon pairs can be full, fused exons or can be fused fragments of exons with a splice junction present. Preferred exon-exon pairs comprising exon fragments may be made from at least two exons, one of which comprises an operable splice donor site and the other of which comprises an operable splice acceptor site and which both are operatively linked by an intron.

Particularly preferred embodiments in this respect, moreover, are polynucleotides which encode polypeptides which retain substantially the same biological function or activity as the mature polypeptides encoded by the gDNA of FIG. 1 [SEQ ID NOs:1-18].

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there is at least 95% and preferably at least 97% identity between the sequences. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5×Denhardt's solution, 10 % dextran sulfate, and 20 microgram/ml denatured, sheared salmon sperm DNA; followed by washing the filters in 0.1×SSC at about 65° C. Thus, the present invention also includes polynucleotides obtainable by screening an appropriate library under stingent hybridization conditions with a labeled probe having the sequence of SEQ ID NOs:1-17 fragments thereof.

As discussed additionally herein regarding polynucleotide assays of the invention, for instance, polynucleotides of the invention as discussed above, may be used as a hybridization probe for cDNA and genomic DNA to isolate full-length cDNAs and genomic clones encoding HFGAN72 receptors and to isolate cDNA and genomic clones of other genes that have a high sequence similarity to the HFGAN72 receptor gene. Such probes generally will comprise at least 15 bases. Preferably, such probes will have at least 30 bases and may have at least 50 bases. Particularly preferred probes will have at least 30 bases and will have 50 bases or less.

For example, the coding region of the HFGAN72 receptor gene may be isolated by screening using the known DNA sequence to synthesize an oligonucleotide probe. A labeled oligonucleotide having a sequence complementary to that of a gene of the present invention is then used to screen a library of human cDNA, genomic DNA or mRNA to determine which members of the library the probe hybridizes to.

The polynucleotides and polypeptides of the present invention may be employed as research reagents and materials for discovery of treatments and diagnostics to human disease, as further discussed herein relating to polynucleotide assays, inter alia.

The polynucleotides may encode a polypeptide which is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may facilitate protein trafficking, may prolong or shorten protein half-life or may facilitate manipulation of a protein for assay or production, among other things. As generally is the case in situ, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In sum, a polynucleotide of the present invention may encode a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

Deposited materials

A deposit containing a human HFGAN72 receptor gDNA has been deposited with the American Type Culture Collection, as noted above. Also as noted above, the gDNA deposit is referred to herein as “the deposited clone” or as “the gDNA of the deposited clone”.

The deposited clone was deposited with the American Type Culture Collection, 10801 University Blvd., Manassas, Va. 20110-2209, USA, on Jul. 2, 1998, and assigned ATCC Deposit Number 98806.

The deposited material is a bacterial clone in E. coli DH10B that contains the full-length human HFGAN72 receptor gDNA, referred to as “pBleoBAC11-102K2”, ATCC Designation #98806, upon deposit.

The deposit has been made under the terms of the Budapest Treaty on the international recognition of the deposit of micro-organisms for purposes of patent procedure. The strain will be irrevocably and without restriction or condition released to the public upon the issuance of a patent. The deposit is provided merely as a convenience to those of skill in the art and is not an admission that a deposit is required for enablement, such as that required under 35 U.S.C. §112.

The sequence of the polynucleotides contained in the deposited material, as well as the amino acid sequence of the polypeptide encoded thereby, are controlling in the event of any conflict with any description of sequences herein.

A license may be required to make, use or sell the deposited materials, and no such license is hereby granted.

Polypeptides

The present invention further relates to the human HFGAN72X receptor polypeptide which has the deduced amino acid sequence set forth in FIG. 2 [SEQ ID NO:19]. Furthermore, the present invention further relates to the human HFGAN72Y receptor polypeptide which has the deduced amino acid sequence set forth in FIG. 3 [SEQ ID NO:20]. Also provided are polypeptides encoded by the HFGAN72 receptor gDNA comprising missense or nonsense mutations, or those polypeptides encoded by unspliced or partially spliced hnRNAs which still comprise at least one intron, particularly those polypeptides which are naturally found in cells, especially human cells. Frameshift mutations have been shown to be associated with disease (Hol, et al. Journal of Medical Genetics, 1995, 32 (1), 52-56).

Particularly preferred embodiments of the invention are polypeptides encoded by differentially spliced polynucleotides, which polypeptides function in cells, especially those which have a biological activity of HFGAN72 receptors, most especially those expressed in human cells.

Still further preferred embodiments of the invention are polypeptides encoded by polynucleotides comprising exon polynucleotide sequences, particularly polynucleotides comprising HFGAN72 receptor exons 1, 2, 3, 4, 5, 6, 7, or 8, having the exon polynucleotide sequences set out in FIG. 1 [SEQ ID NOs:2, 4, 6, 8, 10, 12, 14, and 17], or variants, close homologs, derivatives and analogs thereof, as described above, and polypeptides encoded by polynucleotides which are complementary to such polynucleotides. Other preferred embodiments of the invention are polypeptides encoded by polynucleotides comprising comprising HFGAN72 receptor exons 1, 2, 3, 4, 5, 6, 7, or 8, having the exon polynucleotide sequences set out in FIG. 1 [SEQ ID NOs:2, 4, 6, 8, 10, 12, 14, and 17], operatively liked to the intron of a gene other then HFGAN72 receptors, or joined to an exon of another gene.

The invention also relates to fragments, analogs and derivatives of these polypeptides. The terms “fragment,” “derivative” and “analog” when referring to the polypeptides encoded by the exons of the gDNA of FIG. 1 [SEQ ID NOs:2, 4, 6, 8, 10, 12, 14, and 17], which retains essentially the same biological function or activity as such polypeptide. Thus, an analog includes a proprotein which can be activated by cleavage of the proprotein portion to produce an active mature polypeptide.

The polypeptide of the present invention may be a recombinant polypeptide, a natural polypeptide or a synthetic polypeptide. In certain preferred embodiments, it is a recombinant polypeptide.

The fragment, derivative or analog of the polypeptides encoded by the gDNA set forth in FIG. 1 [SEQ ID NOs:1-17] may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such fragments, derivatives and analogs are deemed to be within the scope of those skilled in the art from the teachings herein.

Among the particularly preferred embodiments of the invention in this regard are polypeptides encoded by the polynucleotides of HFGAN72 receptors set out in FIG. 1 [SEQ ID NOs:2, 4, 6, 8, 10, 12, 14, and 17], variants, analogs, derivatives and fragments thereof, and variants, analogs and derivatives of the fragments. Alternatively, particularly preferred embodiments of the invention in this regard are polypeptides having the amino acid sequence of the HFGAN72 receptors set forth in FIGS. 2 and 3 [SEQ ID NOs:19 and 20], variants, analogs, derivatives and fragments thereof, and variants, analogs and derivatives of the fragments.

Among preferred variants are those that vary from a reference by conservative amino acid substitutions. Such substitutions are those that substitute a given amino acid in a polypeptide by another amino acid of like characteristics. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Further particularly preferred in this regard are variants, analogs, derivatives and fragments, and variants, analogs and derivatives of the fragments, having the amino acid sequence of the HFGAN72 receptor polypeptide of the gDNA set forth in FIG. 1 [SEQ ID NOs:1-18], in which several, a few, 5 to 10, 1 to 5, 1 to 3, 2, 1 or no amino acid residues are substituted, deleted or added, in any combination. Especially preferred among these are silent substitutions, additions and deletions, which do not alter the properties and activities of the HFGAN72 receptor. Also especially preferred in this regard are conservative substitutions. Most highly preferred is the polypeptide having the amino acid sequence encoded by exons 1-7 of FIG. 1 [SEQ ID NOs:2, 4, 6, 8, 10, 12, and 14], as well as the polypeptide encoded by exons 1-6 and 8 of FIG. 1 [SEQ ID NOs:2, 4, 6, 8, 10, 12, and 17] without substitutions.

The polypeptides and polynucleotides of the present invention are preferably provided in an isolated form, and preferably are purified to homogeneity.

The polypeptides of the present invention include the polypeptide encoded by at least one of the exons of exons 1, 2, 3, 4, 5, 6, 7, or 8 (in particular the mature polypeptide) as well as polypeptides which have at least a 95% identity to the polypeptide encoded by at least one of the exons of exons 1, 2, 3, 4, 5, 6, 7, or 8, and more preferably, at least a 97% identity to the polypeptide encoded by at least one of the exons of exons 1, 2, 3, 4, 5, 6, 7, or 8, and, still more preferably, at least a 99% identity to the polypeptide encoded by at least one of the exons of exons 1, 2, 3, 4, 5, 6, 7, or 8 and also include portions of such polypeptides with such portion of the polypeptide generally containing at least 30 amino acids and more preferably at least 50 amino acids.

As known in the art “similarity” between two polypeptides is determined by comparing the amino acid sequence and its conserved amino acid substitutes of one polypeptide to the sequence of a second polypeptide.

Fragments or portions of the polypeptides of the present invention may be employed for producing the corresponding full-length polypeptide by peptide synthesis; therefore, the fragments may be employed as intermediates for producing the full-length polypeptides. Fragments or portions of the polynucleotides of the present invention may be used to synthesize full-length polynucleotides of the present invention.

Preferred variants of polynucleotides of the present invention include splice variants, allelic variants, and polymorphisms, including polynucleotides having one or more single nucleotide polymorphisms (SNPs).

A particularly preferred embodiment of the invention is the genomic polynucleotide sequence of the HFGAN72 receptor variant set forth in FIG. 4 [SEQ ID NOs:21-22], as well as the encoded HFGAN72X variant polypeptide sequence set forth in FIG. 6 [SEQ ID NO:24].

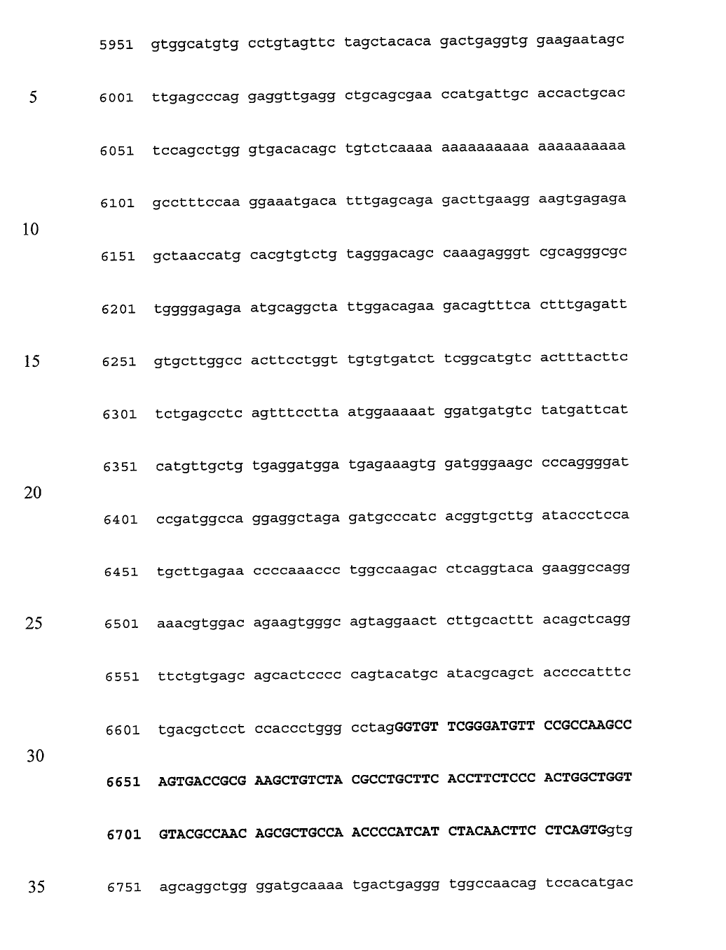

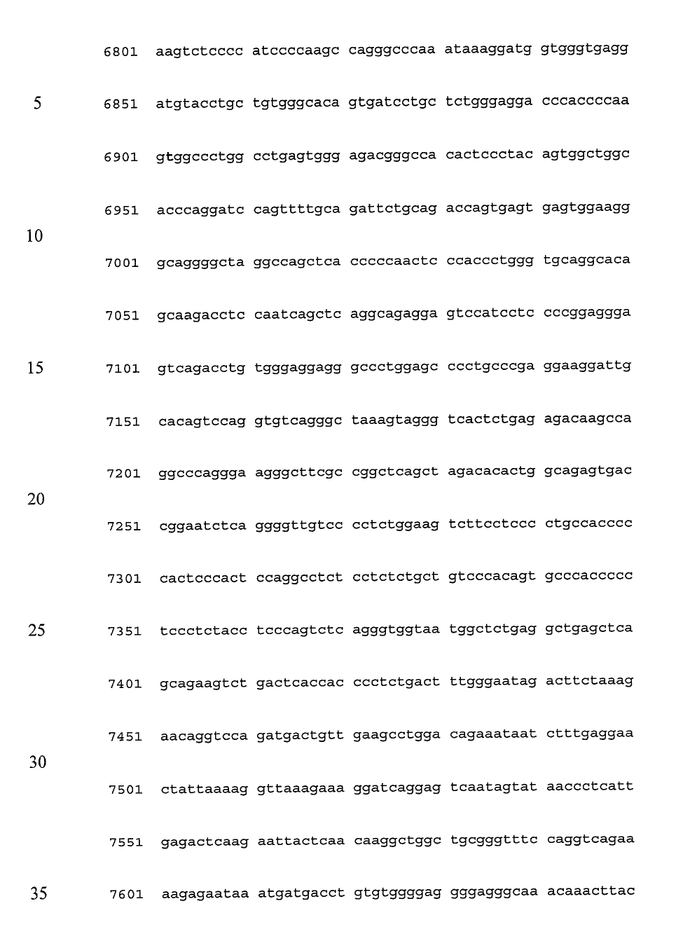

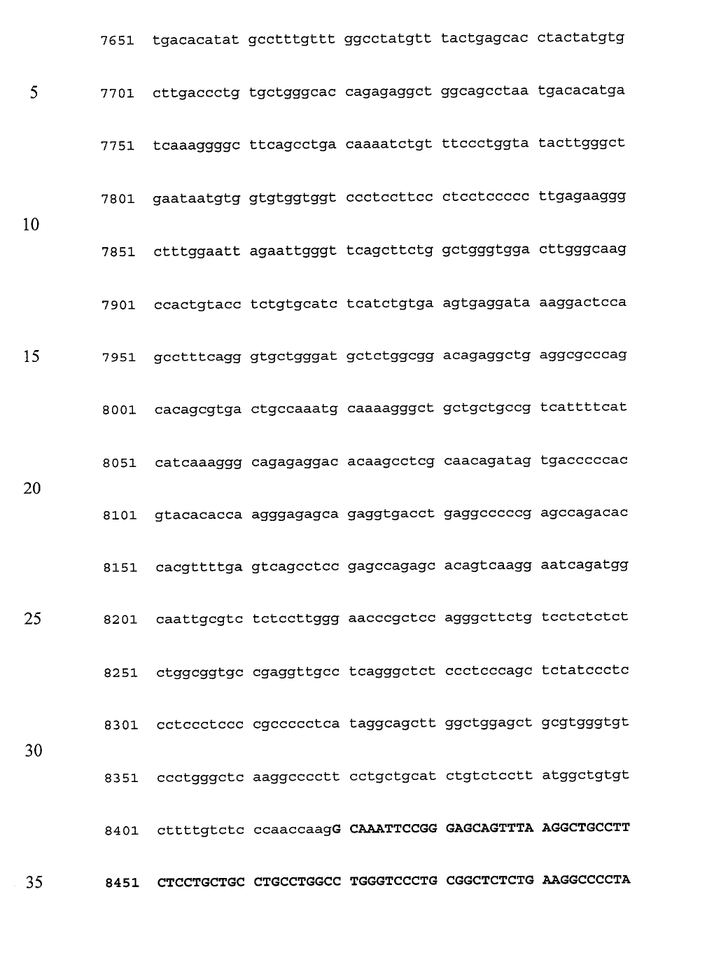

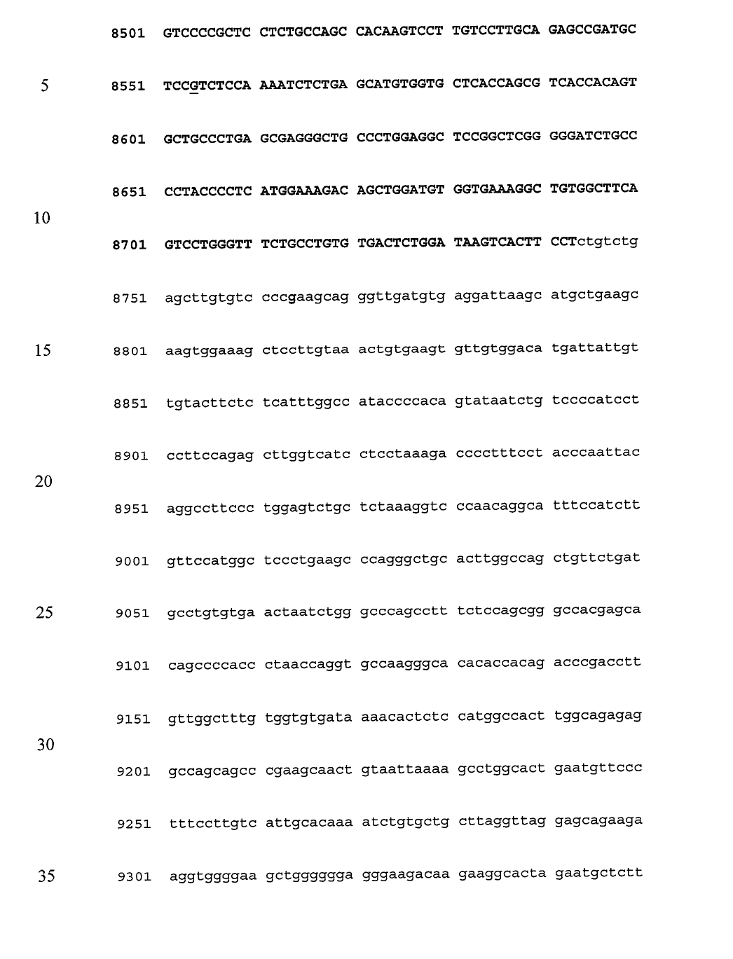

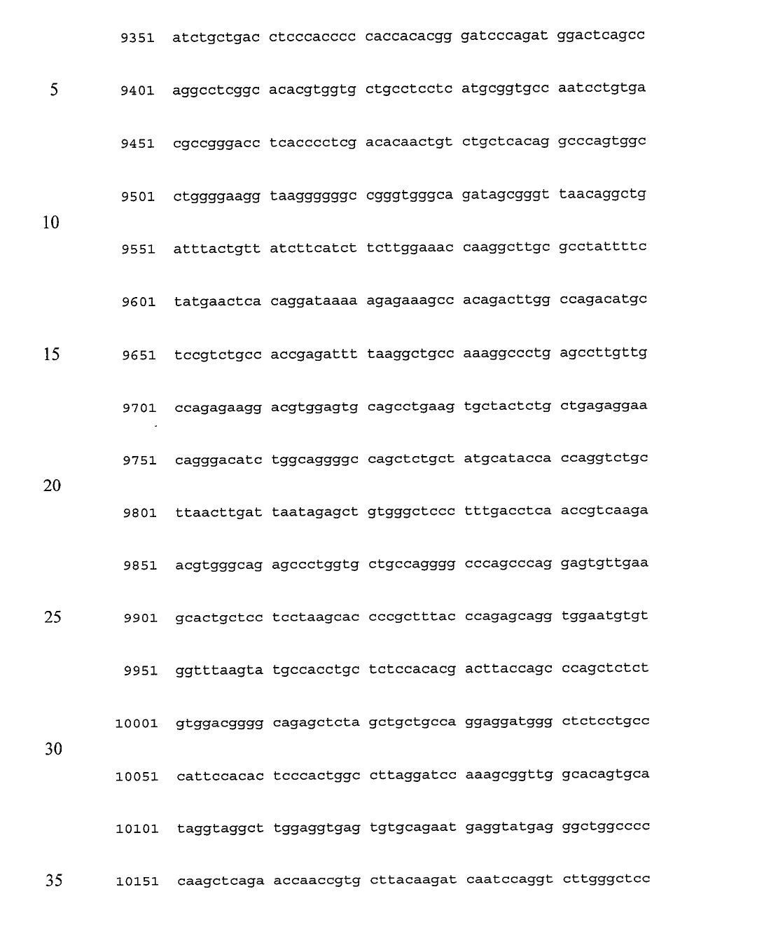

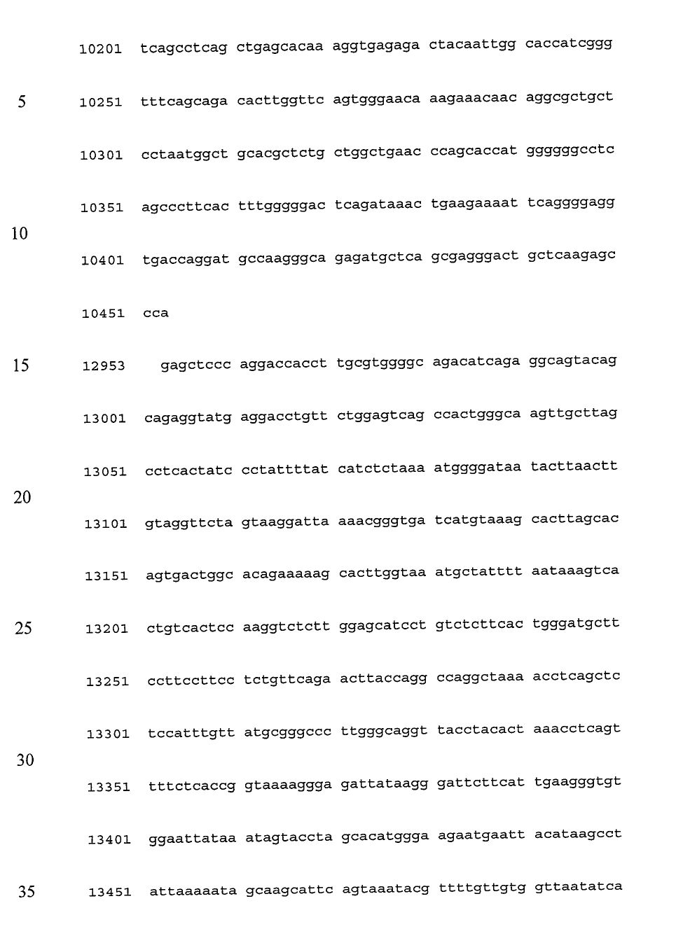

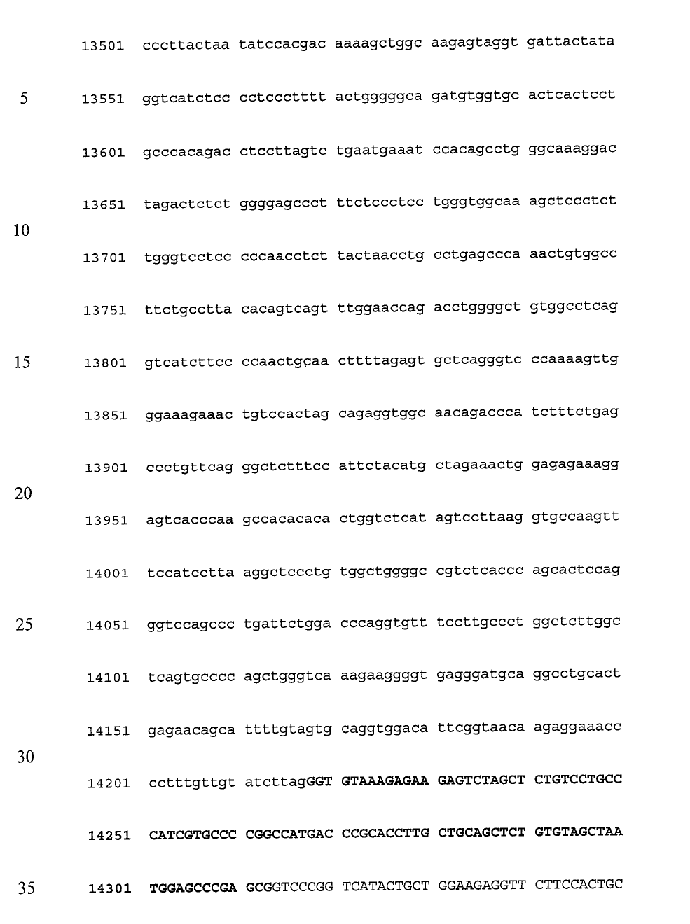

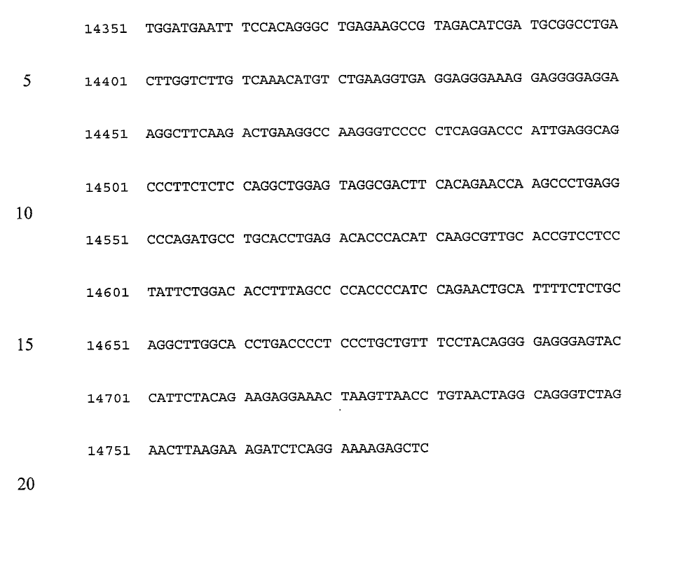

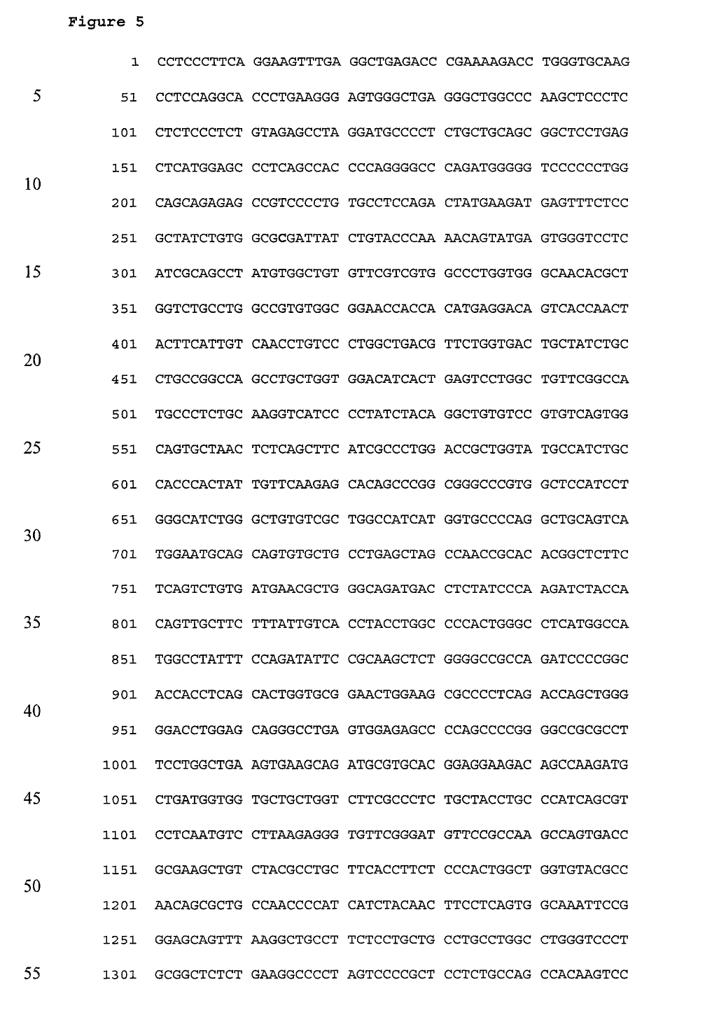

A variant encompassed by the instant invention may by produced by SNPs within the genomic polynucleotide sequence of the HFGAN72 receptors. Such SNPs will either be silent, such that no amino acid change occurs, or will result in amino acid changes in either of the encoded HFGAN72X or HFGAN72Y amino acid sequences set forth in FIGS. 2 and 3 [SEQ ID NOs:19 and 20], respectively. In an alternative embodiment of the invention, SNP occurs at residue 931 of Exon 1 of the genomic polynucleotide sequence of the HFGAN72 receptors set forth in FIG. 1 [SEQ ID NOs:1-18], such that the T at this position may be replaced with an A, C, or G residue. In a particularly preferred embodiment of the invention, the T at residue 931 of the HFGAN72 receptor gDNA is replaced with a C residue, which results in a silent amino acid change of arginine to arginine, as shown in FIG. 4 [SEQ ID NOs:21-22]. In another alternative embodiment of the invention, a SNP occurs at residue 8554 of Exon 7 of the genomic polynucleotide sequence of the HFGAN72 receptors set forth in FIG. 1 [SEQ ID NOs:1-18], such that the A at this position may be replaced with a C, G, or T residue. In a particularly preferred embodiment of the invention, the A at residue 8554 of the genomic polynucleotide sequence of the HFGAN72 receptors is replaced with a G residue, which results in a change in the encoded amino acid sequence from isoleucine to valine, as shown in FIGS. 5 [SEQ ID NOs:23] and 6 [SEQ ID NO:24].

SNPs may also occur in either the 5′ or 3′ UTR, as well as in any of the intronic regions of the genomic polynucleotide sequence of the HFGAN72 receptors of the present invention. In an alternative embodiment of the invention, a SNP occurs at residue 6902 of Intron 6 of the genomic polynucleotide sequence of the HFGAN72 receptors set forth in FIG. 1 [SEQ ID NOs:1-18], such that the c residue at this position can be replaced with an a, g, or t residue. In a particularly preferred embodiment of the invention, the c at residue 6902 of Intron 6 of the genomic polynucleotide sequence of the HFGAN72 receptors is replaced with a t residue, as shown in FIG. 4 [SEQ ID NOs:21-22]. In yet another alternative embodiment of the invention, a SNP occurs at residue 8764 of Intron 7 of the genomic polynucleotide sequence of the HFGAN72 receptors set forth in FIG. 1 [SEQ ID NOs:1-18], such that the t residue at this position can be replaced with an a, c, or g residue. In a particularly preferred embodiment of the invention, the t at residue 8764 of Intron 7 of the genomic polynucleotide sequence of the HFGAN72 receptors is replaced with a g residue, as shown in FIG. 4 [SEQ ID NOs:21-22].

Fragments

Also among preferred embodiments of this aspect of the present invention are polypeptides comprising fragments of HFGAN72 receptors, most particularly fragments of the HFGAN72 receptors having the amino acids sequences set forth in FIGS. 2 and 3 [SEQ ID NOs:19 and 20] and exon fragments or variants and derivatives of these HFGAN72 receptors.

In this regard a fragment is a polypeptide having an amino acid sequence that entirely is the same as part but not all of the amino acid sequence of the aforementioned HFGAN72 receptor polypeptides and variants or derivatives thereof.

Such fragments may be “free-standing,” i.e., not part of or fused to other amino acids or polypeptides, such as, for example, an exon, or they may be comprised within a larger polypeptide of which they form a part or region. When comprised within a larger polypeptide, the presently discussed fragments most preferably form a single continuous region. However, several fragments may be comprised within a single larger polypeptide. For instance, certain preferred embodiments relate to a fragment of a HFGAN72 receptor polypeptide of the present comprised within a precursor polypeptide designed for expression in a host and having heterologous pre and pro-polypeptide regions fused to the amino terminus of the HFGAN72 receptor fragment and an additional region fused to the carboxyl terminus of the fragment. Therefore, fragments in one aspect of the meaning intended herein, refers to the portion or portions of a fusion polypeptide or fusion protein derived from HFGAN72 receptor.

As representative examples of polypeptide fragments of the invention, there may be mentioned those which are encoded by the polynucleotide sequence comprising HFGAN72 receptor exon 1, 2, 3, 4, 5, 6, 7 or 8, having the exon or intron 1, 2, 3, 4, 5, 6 or 7 polynucleotide sequences respectively as set out in FIG. 1 [SEQ ID NOs:2, 4, 6, 8, 10, 12, 14, and 17], or variants, close homologs, derivatives and analogs thereof, as described above, and polypeptides encoded by polynucleotides which are complementary to such polynucleotides.

In this context about includes the particularly recited range and ranges larger or smaller by several, a few, 5, 4, 3, 2 or 1 amino acid at either extreme or at both extremes. For instance, about 65-90 amino acids in this context means a polypeptide fragment of 65 plus or minus several, a few, 5, 4, 3, 2 or 1 amino acids to 90 plus or minus several a few, 5, 4, 3, 2 or 1 amino acid residues, i.e., ranges as broad as 65 minus several amino acids to 90 plus several amino acids to as narrow as 65 plus several amino acids to 90 minus several amino acids.

Highly preferred in this regard are the recited ranges plus or minus as many as 5 amino acids at either or at both extremes. Particularly highly preferred are the recited ranges plus or minus as many as 3 amino acids at either or at both the recited extremes. Especially particularly highly preferred are ranges plus or minus 1 amino acid at either or at both extremes or the recited ranges with no additions or deletions. Most highly preferred of all in this regard are fragments encoded by each of the exons of HFGAN72 receptors.

Among especially preferred fragments of the invention are truncation mutants of HFGAN72 receptors. Truncation mutants include HFGAN72 receptor polypeptides having the amino acid sequence encoded by the exons of FIG. 1 [SEQ ID NOs:2, 4, 6, 8, 10, 12, 14, and 17], or of variants or derivatives thereof, except for deletion of a continuous series of residues (that is, a continuous region, part or portion) that includes the amino terminus, or a continuous series of residues that includes the carboxyl terminus or, as in double truncation mutants, deletion of two continuous series of residues, one including the amino terminus and one including the carboxyl terminus. Fragments having the size ranges set out about also are preferred embodiments of truncation fragments, which are especially preferred among fragments generally.

Also preferred in this aspect of the invention are fragments characterized by structural or functional attributes of HFGAN72 receptors. Preferred embodiments of the invention in this regard include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of HFGAN72 receptors.

Certain preferred regions include Garnier-Robson alpha-regions, beta-regions, turn-regions and coil-regions, Chou-Fasman alpha-regions, beta-regions and turn-regions, Kyte-Doolittle hydrophilic regions and hydrophilic regions, Eisenberg alpha and beta amphipathic regions, Karplus-Schulz flexible regions, Emini surface-forming regions and Jameson-Wolf high antigenic index regions.

Among highly preferred fragments in this regard are those that comprise regions of HFGAN72 receptors that combine several structural features, such as several of the features set out above. In this regard, the exon sequences of FIG. 1 [SEQ ID NOs:2, 4, 6, 8, 10, 12, 14, and 17], which all are characterized by encoding amino acid compositions highly characteristic of turn-regions, hydrophilic regions, flexible-regions, surface-forming regions, and high antigenic index-regions, are especially highly preferred regions. Such regions may be comprised within a larger polypeptide or may be by themselves a preferred fragment of the present invention, as discussed above. It will be appreciated that the term “about” as used in this paragraph has the meaning set out above regarding fragments in general.

Further preferred regions are those that mediate activities of HFGAN72 receptors. Most highly preferred in this regard are fragments that have a chemical, biological, antigenic or other activity of HFGAN72 receptors, including those with a similar activity or an improved activity, or with a decreased undesirable activity.

It will be appreciated that the invention also relates to, among others, polynucleotides encoding the aforementioned fragments, polynucleotides that hybridize to polynucleotides encoding the fragments, particularly those that hybridize under stringent conditions, and polynucleotides, such as PCR primers, for amplifying polynucleotides that encode the fragments. In these regards, preferred polynucleotides are those that correspondent to the preferred fragments, as discussed above.

Other preferred polynucleotides are genetic elements of HFGAN72 receptors, including, but not being limited to, a polyadenylation region, enhancers, a promoter, a cap site introns, exons, and splice sites (references describing these elements include, Darnel, J. et al., Molecular Cell Biology, second edition, W. H. Freeman, New York (1990); Watson, J. D., et al. Molecular Biology of the Gene, Benjamin/Cummings Pub., Menlo Park, Calif., (1987)).

Untranslated regions contain many elements important in regulating gene expression. Mutations and markers in these regions have also been associated with disease (Ozawa T, et al., European Journal of Immunogenetics, April 1995, 22 (2), 163-169). A preferred embodiment of the invention is the 5′ UTR, particularly the sequence set forth in FIG. 1 [SEQ ID NO:1]. Mutations and markers in the 5′ UTR have been associated with disease (Carlock L, et al, Human Genetics, April 1994, 93 (4), 457-459). A particularly preferred polynucleotide is an enhancer and promoter in the 5′ UTR region of the HFGAN72 receptor gDNA. Enhancers are often found in the 5′ UTR and upregulate gene expression (see Miller, et al., Biotechniques 7: 980-990 (1989) for a general reference on promoters). The enhancer of the present invention can be operatively fused to heterologous genes to upregulate gene expression. A particularly preferred polynucleotide is the enhancer promoter having the sequence set forth in FIG. 1 [SEQ ID NO:1]. Transcription factors are often associated with the enhancer and promoter and act to modulate the function of these regions and binding sites for these factors have been described (Faisst, et al., Nucleic Acids Research, Vol. 20, No. 1, pp. 3-26, 1991; Smale, Stephen T., Transcription: Mechanisms and Regulation, Raven Press, Ltd. pp. 63-81 (1994)). These sites bind such factors as, for example, Sp1, Ap1, and Ap3 which are involved in transcription initiation (Faisst, et al., Nucleic Acids Research, Vol. 20, No. 1, pp. 3-26, 1991). The promoter is particularly useful for the study of the control of HFGAN72 receptor gene expression, particularly as a region to be probed to diagnose disease. Vitamin D response elements have been found in the 5′ UTR of known genes (Kahlen, et al., Biochemical & Biophysical Research Communications, Vol. 202, No. 3, pp. 1366-1372 (1994); Darwish, et al., Critical Reviews in Eukaryotic Gene Expression, 3(2):89-116 (1993); Carlberg, Eur. J. Biochem. 231, pp. 517-527 (1995); Ohyama, et al., J. Biol. Chem., Vol. 269, No. 14, pp. 10545-10550 (1994)). Portions of vitamin D (“vD half sites”) responsive elements and calcium ion responsive elements (“Ca half pairs”) are present in the 3′ UTR sequence as set forth in FIG. 1 [SEQ ID NO:18]. Such sites have been described (Katz, et al., J. Biol. Chem., Vol.270, No. 10, pp.5238-5242 (1995)). Promoter factor binding sites found in the promoter and enhancer region and provided in the invention are also found in HFGAN72 receptor introns.

A further preferred embodiment of the invention is the promoter region of HFGAN72 receptors (FIG. 1 [SEQ ID NO:1]). Functional promoter region sequences have been described (Corden, et al., Science, 209, pp. 1406-1414 (1990)). Mutations in the TATA box region of promoters have been shown to be associated with disease (Peltoketo, et al., Genomics, 1994, 23 (1), 250-252).

The 3′ untranslated region of HFGAN72 receptor is a preferred polynucleotide of the invention, especially that polynucleotide set forth in FIG. 1 [SEQ ID NO:18]. Mutations in the 3′ UTR have been associated with disease (Saito, et al., Journal of the American Society of Nephrology, 1994, 4 (9), 1649-1653; Payne, et al., Human Molecular Genetics, 1994, 3 (2), 390). The polyadenylation region set forth in FIG. 1 [SEQ ID NO:18] is also a preferred polynucleotide of the 3′ UTR. The polyadenylation region comprises two copies of the canonical polyadenylation hexanucleotide, AATAAA. The polyadenylation region can be used, for example, in expression vectors to mediate mRNA 3′ end formation (see, for example Gil, et al., Nature 312:473474 (London) (1984)).

Other particularly preferred polynucleotides of the invention are the splice sites, including, but not limited to the splice donors, splice acceptors and the splice branchpoint. Splice junctions formation is essential for the proper creation of an open reading frame (Mount, Stephen, M., Department of Molecular Biophysics and Biochemistry, Yale University, Sterling Hall of Medicine, New Haven, Conn., USA, IRL Press Limited, London, pp. 459-472 (1981)). Diseases associated with the improper formation of the splice junction are known.

Introns comprise elements important in gene expression and in the formation of mature mRNA. Mutations and markers in introns have been shown to be associated with diseases (Peral, et al., Human Molecular Genetics, April 1995, 4 (4), 569-574; Chrysogelos, Nucleic Acids Research, 1993, 21 (24), 5736-5741; Ameis, Journal of Lipid Research, 1995, 36 (2), 241-250). The splice junctions have also been shown to be associated with disease (Ameis, et al., Journal of Lipid Research, February 1995, 36 (2), 241-250; Petrini, et al., Journal of Immunology, 1994, 152 (1), 176-183; Kleiman, et al., Human Genetics, 1994, 94 (3), 279-282). Alternative splicing and cryptic splice sites selection also have been shown to be associated with disease (Arakawa, et al, Human Molecular Genetics, 1994, 3 (4), 565-568; Tieu, et al., Human Mutation, 1994, 3 (3), 333-336; Reale, et al., Cancer Research, 1994, 54 (16), 4493-4501). Introns may also comprise enhancer elements as part of their sequence.

Preferred embodiments of the invention are the HFGAN72 receptor introns, particularly those introns having the sequences set forth in FIG. 1 [SEQ ID NOs:3, 5, 7, 9, 11, 13, 15, and 16]. Polymorphisms in the introns can serve as markers for disease following linkage analysis. Moreover, genetic analyses described herein can be used to locate mutations in the introns associated with and/or causing disease.

Another preferred embodiment is a HFGAN72 receptor intronic enhancer.

Further preferred embodiments of the invention are the HFGAN72 receptor exons, particularly those exons having the sequences set forth in FIG. 1 [SEQ ID NOs:2, 4, 6, 8, 10, 12, 14, and 17]. Polymorphisms in the exons can serve as markers for disease following linkage analysis. Moreover, genetic analyses described herein can be used to locate mutations in the exons associated with and/or causing disease. Methods to detect mutations are known in the art and are generally described in Mashal, et al., Current Opinions in Genetics & Development, 1996, 6: 275-280. In this article, Mashal, et al., describe techniques to identify mutations at multiple potential sites within segments of DNA for which the normal sequence is known. Among the techniques described are conformation-based techniques, base mismatch recognition, and DNA sequence analysis. Id.

Polynucleotide fragments of the invention can be used to create ribozymes that inhibit the expression of the HFGAN72 receptor gene. General methods for the construction of ribozyme constructs are known in the art (Stram, et al., Virus Genes, 1995, 9 (2), 155-159). Skilled artisans can readily adapt these methods using the novel fragments of the invention to create novel ribozyme constructs. Preferred ribozyme constructs comprise sequences which are complementary to the transcribed control elements of the HFGAN72 receptor gene, particularly polynucleotides that are complementary to the 5′ untranslated region, splice junctions, and 3′ untranslated region, especially the polyadenylation region.

The fragments of the invention, particularly regions in the untranslated region, the promoter and introns are useful as diagnostic probes for disease, particularly infections such as bacterial, fungal, protozoan and viral infections, particularly infections caused by HIV-1 or HIV-2; cancers; diabetes; asthma; Parkinson's disease; both acute and congestive heart failure; hypotension; hypertension; urinary retention; osteoporosis; angina pectoris; myocardial infarction; ulcers; asthma; allergies; benign prostatic hypertrophy; chronic renal failure; renal disease; impaired glucose tolerance; seizure disorder; depression; anxiety; obsessive compulsive disorder; affective neurosis/disorder; depressive neurosis/disorder; anxiety neurosis; dysthymic disorder; behavior disorder; mood disorder; schizophrenia; psychosexual dysfunction; sex disorder; sexual disorder; disturbed biological and circadian rhythms; feeding disorders, such as anorexia, bulimia, cachexia, and obesity; Cushing's syndrome/disease; basophil adenoma; prolactinoma; hyperprolactinemia; hypopituitarism; hypophysis tumor/adenoma; hypothalamic diseases; Froehlich's syndrome; adenohypophysis disease; hypophysis disease; hypophysis tumor/adenoma; pituitary growth hormone; adenohypophysis hypofunction; adrenohpophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, heart and lung diseases, mental illness, and addictions; migraine; hyperalgesia; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labour pain; cancer pain; post-chemotherapy pain; post-stroke pain; post-operative pain; neuralgia; and tolerance to narcotics or withdrawal from narcotics; sleep disorders; sleep apnea; narcolepsy; insomnia; parasomnia; jet-lag syndrome; and other neurodegenerative disorders, which includes nosological entities such as disinhibition-dementia-parkinsonism-amyotrophy complex; pallido-ponto-nigral degeneration; and dyskinesias, such as Huntington's disease or Gilles dela Tourett's syndrome, among others. Moreover, markers for disease can be located in regions of the HFGAN72 receptor gene, particularly untranslated regions, which are useful with the diagnostic methods of the invention.

Vectors, host cells, expression

The present invention also relates to vectors which include polynucleotides of the present invention, host cells which are genetically engineered with vectors of the invention and the production of polypeptides of the invention by recombinant techniques.

Host cells can be genetically engineered to incorporate polynucleotides and express polypeptides of the present invention. For instance, polynucleotides may be introduced into host cells using well known techniques of infection, transduction, transfection, transvection and transformation. The polynucleotides may be introduced alone or with other polynucleotides. Such other polynucleotides may be introduced independently, co-introduced or introduced joined to the polynucleotides of the invention.

Thus, for instance, polynucleotides of the invention may be transfected into host cells with another, separate, polynucleotide encoding a selectable marker, using standard techniques for co-transfection and selection in, for instance, mammalian cells. In this case the polynucleotides generally will be stably incorporated into the host cell genome.

Alternatively, the polynucleotides may be joined to a vector containing a selectable marker for propagation in a host. The vector construct may be introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it may be packaged in vitro or introduced into a packaging cell and the packaged virus may be transduced into cells. A wide variety of techniques suitable for making polynucleotides and for introducing polynucleotides into cells in accordance with this aspect of the invention are well known and routine to those of skill in the art. Such techniques are reviewed at length in Sambrook, et al., cited above, which is illustrative of the many laboratory manuals that detail these techniques.

In accordance with this aspect of the invention the vector may be, for example, a plasmid vector, a single or double-stranded phage vector, a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsidated virus by well known techniques for infection and transduction. Viral vectors may be replication competent or replication defective. In the latter case viral propagation generally will occur only in complementing host cells.

Preferred among vectors, in certain respects, are those for expression of polynucleotides and polypeptides of the present invention. Generally, such vectors comprise cis-acting control regions effective for expression in a host operatively linked to the polynucleotide to be expressed. Appropriate trans-acting factors either are supplied by the host, supplied by a complementing vector or supplied by the vector itself upon introduction into the host.

In certain preferred embodiments in this regard, the vectors provide for specific expression. Such specific expression may be inducible expression or expression only in certain types of cells or both inducible and cell-specific. Particularly preferred among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art.

The engineered host cells can be cultured in conventional nutrient media, which may be modified as appropriate for, inter alia, activating promoters, selecting transformants or amplifying genes. Culture conditions, such as temperature, pH and the like, previously used with the host cell selected for expression generally will be suitable for expression of polypeptides of the present invention as will be apparent to those of skill in the art.

A great variety of expression vectors can be used to express a polypeptide of the invention. Such vectors include chromosomal, episomal and virus-derived vectors, e.g., vectors derived from bacterial plasmids, from bacteriophage, from yeast episomes, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, all may be used for expression in accordance with this aspect of the present invention. Generally, any vector suitable to maintain, propagate or express polynucleotides to express a polypeptide in a host may be used for expression in this regard.

The appropriate DNA sequence may be inserted into the vector by any of a variety of well-known and routine techniques. In general, a DNA sequence for expression is joined to an expression vector by cleaving the DNA sequence and the expression vector with one or more restriction endonucleases and then joining the restriction fragments together using T4 DNA ligase. Procedures for restriction and ligation that can be used to this end are well known and routine to those of skill. Suitable procedures in this regard, and for constructing expression vectors using alternative techniques, which also are well known and routine to those skill, are set forth in great detail in Sambrook, et al., cited elsewhere herein.

The DNA sequence in the expression vector is operatively linked to appropriate expression control sequence(s), including, for instance, a promoter to direct mRNA transcription. Representatives of such promoters include the phage lambda PL promoter, the E. coli lac, trp and tac promoters, the SV40 early and late promoters and promoters of retroviral LTRs, to name just a few of the well-known promoters. It will be understood that numerous promoters not mentioned are suitable for use in this aspect of the invention are well known and readily may be employed by those of skill in the manner illustrated by the discussion and the examples herein.

In general, expression constructs will contain sites for transcription initiation and termination, and, in the transcribed region, a ribosome binding site for translation. The coding portion of the mature transcripts expressed by the constructs will include a translation initiating AUG at the beginning and a termination codon appropriately positioned at the end of the polypeptide to be translated.

In addition, the constructs may contain control regions that regulate as well as engender expression. Generally, in accordance with many commonly practiced procedures, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors preferably contain one or more selectable marker genes to provide a phenotypic trait for selection of transformed host cells. Preferred markers include dihydrofolate reductase or neomycin resistance for eukaryotic cell culture, tetracycline, kanamycin, and ampicillin resistance genes for culturing E. coli and other bacteria.

The vector containing the appropriate DNA sequence as described elsewhere herein, as well as an appropriate promoter, and other appropriate control sequences, may be introduced into an appropriate host using a variety of well known techniques suitable to expression therein of a desired polypeptide. Representative examples of appropriate hosts include bacterial cells, such as E. coli, streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Hosts for a great variety of expression constructs are well known, and those of skill will be enabled by the present disclosure readily to select a host for expressing a polypeptides in accordance with this aspect of the present invention.

More particularly, the present invention also includes recombinant constructs, such as expression constructs, comprising one or more of the sequences described above. The constructs comprise a vector, such as a plasmid or viral vector, into which such a sequence of the invention has been inserted. The sequence may be inserted in a forward or reverse orientation. In certain preferred embodiments in this regard, the construct further comprises regulatory sequences, including, for example, a promoter, operably linked to the sequence. Large numbers of suitable vectors and promoters are known to those of skill in the art, and there are many commercially available vectors suitable for use in the present invention.

The following vectors, which are commercially available, are provided by way of example. Among vectors preferred for use in bacteria are pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. These vectors are listed solely by way of illustration of the many commercially available and well known vectors that are available to those of skill in the art for use in accordance with this aspect of the present invention. It will be appreciated that any other plasmid or vector suitable for, for example, introduction, maintenance, propagation or expression of a polynucleotide or polypeptide of the invention in a host may be used in this aspect of the invention.

Promoter regions can be selected from any desired gene using vectors that contain a reporter transcription unit lacking a promoter region, such as a chloramphenicol acetyl transferase (“CAT”) transcription unit, downstream of restriction site or sites for introducing a candidate promoter fragment; i.e., a fragment that may contain a promoter. As is well known, introduction into the vector of a promoter-containing fragment at the restriction site upstream of the cat gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well known and readily available promoters, but also promoters that readily may be obtained by the foregoing technique, using a reporter gene.

A preferred embodiment of the invention are expression vectors comprising HFGAN72 receptor promoter sequences that function as a promoter. Such vector constructs may be used for targeted gene expression in cells which utilize the HFGAN72 receptor promoter, for example, osteoclasts and macrophages. Any gene of interest can be expressibly linked to the HFGAN72 receptor promoter and expressed in such cells which utilize the HFGAN72 receptor promoter. In this manner genes which immortalize primary eukaryotic cells, such as, for example, SV40 T-Antigen, may be expressibly linked HFGAN72 receptor promoter to immortalize cells, such as, for example, bone cells, including osteoclasts, and macrophages. Certain preferred vectors comprise HFGAN72 receptor expressibly linked to a toxin gene, such as for example, ricin, and are useful in methods for the targeted killing of cell populations that utilize the HFGAN72 receptor promoter for gene expression. Certain other preferred vectors comprise HFGAN72 receptor promoter expressibly linked to a anti-HFGAN72 receptor ribozyme or antisense polynucleotide, which are useful in methods for such targeted killing.

Among known bacterial promoters suitable for expression of polynucleotides and polypeptides in accordance with the present invention are the E. coli lacI and lacZ promoters, the T3 and T7 promoters, the gpt promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

Selection of appropriate vectors and promoters for expression in a host cell is a well known procedure and the requisite techniques for expression vector construction, introduction of the vector into the host and expression in the host are routine skills in the art.

The present invention also relates to host cells containing the above-described constructs discussed above. The host cell can be a higher eukaryotic cell, such as a mammalian cell or insect cell, or a lower eukaryotic cell, such as a yeast cell, or the host cell can be a prokaryotic cell, such as a bacterial cell.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis, et al. BASIC METHODS IN MOLECULAR BIOLOGY, (1986).

Constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al, MOLECULAR CLONING: A LABORATORY MANUAL, 2nd Ed., Cold Spring Harbor Laboratory Press, Cold Spring Harbor, N.Y. (1989).

Generally, recombinant expression vectors for yeast will include origins of replication, a promoter derived from a highly-expressed gene to direct transcription of a downstream structural sequence, and a selectable marker to permit isolation of vector containing cells after exposure to the vector. Among suitable promoters are those derived from the genes that encode glycolytic enzymes such as 3-phosphoglycerate kinase (“PGK”), a-factor, acid phosphatase, and heat shock proteins, among others. Selectable markers include the ampicillin resistance gene of E. coli and the trp1 gene of S. cerevisiae.

Transcription of the DNA encoding the polypeptides of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at bp 100 to 270, the cytomegalovirus early promoter enhancer, the polyoma enhancer on the late side of the replication origin, and adenovirus enhancers.

Polynucleotides of the invention, encoding the heterologous structural sequence of a polypeptide of the invention generally will be inserted into the vector using standard techniques so that it is operably linked to the promoter for expression. The polynucleotide will be positioned so that the transcription start site is located appropriately 5′ to a ribosome binding site. The ribosome binding site will be 5′ to the AUG that initiates translation of the polypeptide to be expressed. Generally, there will be no other open reading frames that begin with an initiation codon, usually AUG, and lie between the ribosome binding site and the initiating AUG. Also, generally, there will be a translation stop codon at the end of the polypeptide and there will be a polyadenylation signal and a transcription termination signal appropriately disposed at the 3′ end of the transcribed region.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a fusion protein, and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, a region may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

Suitable prokaryotic hosts for propagation, maintenance or expression of polynucleotides and polypeptides in accordance with the invention include Escherichia coli, Bacillus subtilis and Salmonella typhimurium. Various species of Pseudomonas, Streptomyces, and Staphylococcus are suitable hosts in this regard. Moreover, many other hosts also known to those of skill may be employed in this regard.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis, USA). These pBR322 “backbone” sections are combined with an appropriate promoter and the structural sequence to be expressed.

Following transformation of a suitable host strain and growth of the host strain to an appropriate cell density, where the selected promoter is inducible it is induced by appropriate means (e.g., temperature shift or exposure to chemical inducer) and cells are cultured for an additional period.

Cells typically then are harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

Various mammalian cell culture systems can be employed for expression, as well. Examples of mammalian expression systems include the COS-7 lines of monkey kidney fibroblast, described in Gluzman, et al, Cell 23: 175 (1981). Other cell lines capable of expressing a compatible vector include for example, the C127, 3T3, CHO, HeLa, human kidney 293 and BHK cell lines.

Mammalian expression vectors will comprise an origin of replication, a suitable promoter and enhancer, and also any necessary ribosome binding sites, polyadenylation sites, splice donor and acceptor sites, transcriptional termination sequences, and 5′ flanking non-transcribed sequences that are necessary for expression. In certain preferred embodiments in this regard DNA sequences derived from the SV40 splice sites, and the SV40 polyadenylation sites are used for required non-transcribed genetic elements of these types.

The HFGAN72 receptor polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Well known techniques for refolding protein may be employed to regenerate active conformation when the polypeptide is denatured during isolation and or purification.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Further illustrative aspects and preferred embodiments of the invention

HFGAN72 receptor polynucleotides and polypeptides may be used in accordance with the present invention for a variety of applications, particularly those that make use of the chemical and biological properties of HFGAN72 receptor. Among these are applications in the detection and treatment of disease, particularly infections such as bacterial, fungal, protozoan and viral infections, particularly infections caused by HIV-1 or HIV-2; cancers; diabetes; ash; Parkinson's disease; both acute and congestive heart failure; hypotension, hypertension; urinary retention, osteoporosis; angina pectoris; myocardial infarction; ulcers; asthma; allergies; benign prostatic hypertrophy; chronic renal failure; renal disease; impaired glucose tolerance; seizure disorder; depression; anxiety; obsessive compulsive disorder; affective neurosis/disorder; depressive neurosis/disorder; anxiety neurosis; dysthymic disorder; behavior disorder; mood disorder; schizophrenia, psychosexual dysfunction; sex disorder; sexual disorder; disturbed biological and circadian rhythms; feeding disorders, such as anorexia, bulimia, cachexia, and obesity; Cushing's syndrome/disease; basophil adenoma; prolactinoma; hyperprolactinemia; hypopituitarism; hypophysis tumor/adenoma; hypothalamic diseases; Froehlich's syndrome; adenohypophysis disease; hypophysis disease; hypophysis tumor/adenoma; pituitary growth hormone; adenohypophysis hypofunction; adrenohpophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, heart and lung diseases, mental illness, and addictions; migraine; hyperalgesia; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labour pain; cancer pain; post-chemotherapy pain; post-stroke pain; post-operative pain; neuralgia; and tolerance to narcotics or withdrawal from narcotics; sleep disorders; sleep apnea; narcolepsy; insomnia; parasomnia; jet-lag syndrome; and other neurodegenerative disorders, which includes nosological entities such as disinhibition-dementia-parkinsonism-amyotrophy complex; pallido-ponto-nigral degeneration; and dyskinesias, such as Huntington's disease or Gilles dela Tourett's syndrome, among others. Additional applications relate to diagnosis and to treatment of disorders of cells, tissues and organisms. These aspects of the invention are illustrated further by the following discussion.

Polynucleotide assays

This invention is also related to the use of the HFGAN72 receptor exons, introns, promoters and polynucleotides to detect complementary polynucleotides such as, for example, as a diagnostic reagent. Detection of a mutated form of HFGAN72 receptor associated with a dysfunction will provide a diagnostic tool that can add or define a diagnosis of a disease or susceptibility to a disease which results from under-expression, over-expression or altered expression of HFGAN72 receptor, such as, for example, bacterial, fungal, protozoan and viral infections, particularly infections such as bacterial, fungal, protozoan and viral infections, particularly infections caused by HIV-1 or HIV-2; cancers; diabetes; asthma; Parkinson's disease; both acute and congestive heart failure, hypotension; hypertension; urinary retention; osteoporosis; angina pectoris; myocardial infarction; ulcers; asthma; allergies; benign prostatic hypertrophy; chronic renal failure; renal disease; impaired glucose tolerance; seizure disorder; depression; anxiety; obsessive compulsive disorder; affective neurosis/disorder; depressive neurosis/disorder; anxiety neurosis; dysthymic disorder; behavior disorder; mood disorder; schizophrenia; psychosexual dysfunction; sex disorder; sexual disorder; disturbed biological and circadian rhythms; feeding disorders, such as anorexia, bulimia, cachexia, and obesity; Cushing's syndrome/disease; basophil adenoma; prolactinoma; hyperprolactinemia; hypopituitarism; hypophysis tumor/adenoma; hypothalamic diseases; Froehlich's syndrome; adenohypophysis disease; hypophysis disease; hypophysis tumor/adenoma; pituitary growth hormone; adenohypophysis hypofunction; adrenohpophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, heart and lung diseases, mental illness, and addictions; migraine; hyperalgesia; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labour pain; cancer pain; post-chemotherapy pain; post-stroke pain; post-operative pain; neuralgia; and tolerance to narcotics or withdrawal from narcotics; sleep disorders; sleep apnea; narcolepsy; insomnia; parasomnia; jet-lag syndrome; and other neurodegenerative disorders, which includes nosological entities such as disinhibition-dementia-parkinsonism-amyotrophy complex; pallido-ponto-nigral degeneration; and dyskinesias, such as Huntington's disease or Gilles dela Tourett's syndrome, among others.

Individuals carrying mutations in the human HFGAN72 receptor gene may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR prior to analysis (Saiki, et al., Nature, 324: 163-166 (1986)). Ligation-mediated amplification may also be used for amplification (Vollach, et al., Nucl. Acids Res. 22: 2507 (1994). RNA or cDNA may also be used in the same ways. As an example, PCR primers complementary to the nucleic acid encoding HFGAN72 receptors can be used to identify and analyze HFGAN72 receptor expression and mutations. For example, deletions and insertions can be detected by a change in size of the amplified product in comparison to the normal genotype. Point mutations can be identified by hybridizing amplified DNA to radiolabeled HFGAN72 receptor RNA or alternatively, radiolabeled HFGAN72 receptor antisense DNA sequences. Perfectly matched sequences can be distinguished from mismatched duplexes by RNase A digestion or by differences in melting temperatures.

Sequence differences between a reference gene and genes having mutations also may be revealed by direct DNA sequencing. In addition, cloned DNA segments may be employed as probes to detect specific DNA segments. The sensitivity of such methods can be greatly enhanced by appropriate use of PCR or another amplification method. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels, with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers, et al., Science, 230: 1242 (1985)).

Sequence changes at specific locations also may be revealed by nuclease protection assays, such as RNase and S1 protection or the chemical cleavage method (e.g., Cotton, et al., Proc. Natl. Acad. Sci., USA, 85: 4397-4401 (1985)).

Thus, the detection of a specific DNA sequence may be achieved by methods such as hybridization, RNase protection, chemical cleavage, direct DNA sequencing or the use of restriction enzymes, (e.g., restriction fragment length polymorphisms (“RFLP”), SSCP and Southern blotting of genomic DNA.

In addition to more conventional gel-electrophoresis and DNA sequencing, mutations also can be detected by in situ analysis.

Chromosome assays

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the gDNA herein disclosed is used to clone genomic DNA of a HFGAN72 receptor gene. This can be accomplished using a variety of well known techniques and libraries, which generally are available commercially. The genomic DNA then is used for in situ chromosome mapping using well known techniques for this purpose. Typically, in accordance with routine procedures for chromosome mapping, some trial and error may be necessary to identify a genomic probe that gives a good in situ hybridization signal.

In some cases, in addition, sequences can be mapped to chromosomes by preparing PCR primers (preferably 15-25 bp) from the gDNA. Computer analysis of the 3′ untranslated region of the gene is used to rapidly select primers that do not span more than one exon in the genomic DNA complicate the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes (e.g., radiation hybrid panels) or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (“FISH”) of a cDNA clone to a metaphase chromosomal spread can be used to provide a precise chromosomal location in one step. This technique can be used with gDNA as short as 50 to as long as 600. For a review of this technique, see Verma, et al., HUMAN CHROMOSOMES: A MANUAL OF BASIC TECHNIQUES, Pergamon Press, New York (1988).

Once a sequence has been mapped to a precise chromosomal location, the physical position of the sequence on the chromosome can be correlated with genetic map data. Such data are found, for example, in V. McKusick, MENDELIAN INHERITANCE IN MAN, available on line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a gDNA precisely localized to a chromosomal region associated with the disease could be one of between 50 and 500 potential causative genes. (This assumes 1 megabase mapping resolution and one gene per 20 kb).

Clinical Genomics

This invention provides methods to determine drug responsiveness of individuals having or suspected of having a HFGAN72 receptor gene mutation or HFGAN72 receptor gene expression abnormality, and also provides reagents to carry out such methods. Individuals may be grouped by their responsiveness to a given compound, particularly drugs, used to treat diseases caused by or associated with a mutation of HFGAN72 receptor gene or HFGAN72 receptor gene expression. Such individuals may be further grouped by detecting different gene mutations or gene expression level variants. In this manner, specific gene mutations and gene expression variants can be readily associated with a certain degree of responsiveness to a compound by an individual). Methods and reagents provided herein can be used to group compound responsiveness by detecting HFGAN72 receptor gene mutations and HFGAN72 receptor gene expression variants. Other methods for grouping individuals by compound responsivness are known to skilled artisans and can be adapted to use the polypeptides and polynucleotides of the invention.

The invention also provides algorithms useful in conjunction with a device or embodied in a composition matter which are useful for the diagnosis of diseases caused by or associated with HFGAN72 receptor or mutants or variants thereof. Preferred algorithms are provided for disease stratification and staging.

Compositions

The invention also relates to compositions comprising the polynucleotides or the polypeptides discussed. Thus, the polypeptides of the present invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a polypeptide of the invention and a pharmaceutically acceptable carrier or excipient. Such carriers may include, but are not limited to, saline, buffered saline, dextrose, water, glycerol, ethanol and combinations thereof. The formulation should suit the mode of administration.

Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

Administration

Polypeptides of the present invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal, intraarticular, or intradermal routes among others.

The pharmaceutical compositions generally are administered in an amount effective for treatment or prophylaxis of a specific indication or indications. In general, the compositions are administered in an amount of at least about 10 mg/kg body weight. Preferably, in most cases, dose is from about 10 mg/kg to about 1 mg/kg body weight, daily. It will be appreciated that optimum dosage will be determined by standard methods for each treatment modality and indication, taking into account the indication, its severity, route of administration, complicating conditions and the like.

Gene therapy

The HFGAN72 receptor polynucleotides, polypeptides, agonists and antagonists that are polypeptides may be employed in accordance with the present invention by expression of such polypeptides in vivo, in treatment modalities often referred to as “gene therapy.”

Thus, for example, cells from a patient may be engineered with a polynucleotide, such as a DNA or RNA, encoding a polypeptide ex vivo, and the engineered cells then can be provided to a patient to be treated with the polypeptide. For example, cells may be engineered ex vivo by the use of a retroviral plasmid vector containing RNA encoding a polypeptide of the present invention. Such methods are well-known in the art and their use in the present invention will be apparent from the teachings herein.

Cells from a patient may also be engineered with a polynucleotide, such as a ribozyme that has been constructed, using well known methods, to inhibit the gene expression of HFGAN72 receptors. Other constructs may also be engineered into a patient's cells to contains antisense stretches of HFGAN72 receptor sequence, using well known methods. Such antisense constructs will inhibit HFGAN72 receptor expression in the patient.

Similarly, cells may be engineered in vivo for expression of a polypeptide in vivo by procedures known in the art. For example, a polynucleotide of the invention may be engineered for expression in a replication defective retroviral vector, as discussed above. The retroviral expression construct then may be isolated and introduced into a packaging cell that is transduced with a retroviral plasmid vector containing RNA encoding a polypeptide of the present invention such that the packaging cell now produces infectious viral particles containing the gene of interest. These producer cells may be administered to a patient for engineering cells in vivo and expression of the polypeptide in vivo. These and other methods for administering a polypeptide of the present invention by such method should be apparent to those skilled in the art from the teachings of the present invention.

Retroviruses from which the retroviral plasmid vectors herein above mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

Such vectors well include one or more promoters for expressing the polypeptide. Suitable promoters which may be employed include, but are not limited to, HFGAN72 receptor promoter, a retroviral LTR, an SV40 promoter, and the human cytomegalovirus (CMV) promoter described in Miller, et al., Biotechniques 7: 980-990 (1989), or any other promoter (e.g., cellular promoters such as eukaryotic cellular promoters including, but not limited to, the histone, RNA polymerase III, and alpha-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide of the present invention will be placed under the control of a suitable promoter. Suitable promoters which may be employed include, but are not limited to, adenoviral promoters, such as the adenoviral major late promoter, or heterologous promoters, such as the cytomegalovirus (CMV) promoter; the rous sarcoma virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs herein above described); the alpha-actin promoter; and human growth hormone promoters. The promoter also may be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, Y-2, Y-AM, PA12, T19-14X, VT-19-17-H2, YCRE, YCRIP, GP⁺E-86, GP⁺ envAm12, and DAN cell lines as described in Miller, A., Human Gene Therapy 1: 5-14 (1990). The vector may be transduced into the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line will generate infectious retroviral vector particles, which include the nucleic acid sequence(s) encoding the polypeptides. Such retroviral vector particles then may be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

### EXAMPLES

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

Certain terms used herein are explained in the foregoing glossary.

An N used herein in a nucleotide sequence refers to an unknown nucleotide or nucleotides.

All examples were or may be carried out using standard techniques, which are well known and routine to those of skill in the art, except where otherwise described in detail. Routine molecular biology techniques of the following examples can be carried out as described in standard laboratory manuals, such as Sambrook, et al., MOLECULAR CLONING: A LABORATORY MANUAL, 2nd Ed.; Cold Spring Harbor Laboratory Press, Cold Spring Harbor, N.Y. (1989), herein referred to as “Sambrook.”

All parts or amounts set out in the following examples are by weight, unless otherwise specified.

Unless otherwise stated size separation of fragments in the examples below was carried out using standard techniques of agarose and polyacrylamide gel electrophoresis (“PAGE”) in Sambrook and numerous other references such as, for instance, by Goeddel, et al., Nucleic Acids Res. 8: 4057 (1980).

Unless described otherwise, ligations were accomplished using standard buffers, incubation temperatures and times, approximately equimolar amounts of the DNA fragments to be ligated and approximately 10 units of T4 DNA ligase (“ligase”) per 0.5 μg of DNA.

### Example 1

### Isolation and Sequencing of Human HFGAN72 Receptor Genomic Clone

A bacterial phage library containing human genomic DNA was screened using HFGAN72 cDNA as a hybridization probe. A single phage that hybridized to the cDNA probe was isolated, DNA was purified, and a portion of the human genomic DNA insert was subcloned into a plasmid vector and its DNA sequence was determined, which is shown in FIG. 1 (SEQ ID NOs:1-18).

It will be clear that the invention may be practiced otherwise than as particularly described in the foregoing description and examples.

Numerous modifications and variations of the present invention are possible in light of the above teachings and, therefore, are within the scope of the appended claims.

All publications, including but not limited to patents and patent applications, cited in this specification are herein incorporated by reference as if each individual publication were specifically and individually indicated to be incorporated by reference herein as though fully set forth

What is claimed is:

1. An isolated polynucleotide comprising a region selected from the group consisting of:
a sequence having at least a 95% identity to sequence selected from the group consisting of: SEQ ID NOs:1-18.

2. An isolated polynucleotide according to claim 1, wherein said region is a genomic DNA.

3. An isolated polynucleotide comprising a sequence selected from the group consisting of:
intron 1, 2, 3, 4, 5, 6 and 7.

4. An isolated polypeptide encoded by a polynucleotide comprising a sequence selected from the group consisting of:
intron 1, 2, 3, 4, 5, 6 and 7.

5. An isolated polynucleotide comprising a HFGAN72 receptor exon.

6. The isolated polynucleotide of claim 5 having the sequence in SEQ ID NOs:2, 4, 5, 8, 10, 12, 14, and 17.

7. An isolated polynucleotide consisting of a sequence selected from the group consisting of:
exon 1, 2, 3, 4, 5, 6, 7 and 8.

8. An isolated polypeptide encoded by a polynucleotide consisting of a sequence selected from the group consisting of:
exon 1, 2, 3, 4, 5, 6, 7, and 8.

9. An expression vector comprising cis-acting control elements effective for expression in a host cell of an operatively linked polynucleotide according to claim 1.

10. An expression vector according to claim 9, wherein said control elements are effective for inducible expression of said polynucleotide in said host cell.

11. A process for making a polypeptide, comprising the step of expressing in a host cell a polynucleotide according to claim 1.

12. A host cell genetically engineered with the vector of claim 9.

13. A process for producing a polypeptide comprising the step of: expressing from the host cell of claim 12 the polypeptide encoded by said DNA.

14. A method for determining a HFGAN72 receptor-encoding polynucleotide in a sample, comprising the steps of:
(a) hybridizing to a sample a probe specific for said polynucleotide under conditions effective for said probe to hybridize specifically to said polynucleotide; and
(b) determining the hybridization of said probe to polynucleotides in said sample, wherein said probe comprises its sequence a region of 20 or more base pairs at least 95% identical to the polynucleotide sequence of SEQ ID NOs:2, 4, 6, 8, 10, 12, 14, and 17.

15. A method for determining a HFGAN72 receptor-encoding polynucleotide in a sample, comprising the steps of:
(a) hybridizing to a sample a probe specific for said polynucleotide under conditions effective for said probe to hybridize specifically to said polynucleotide; and
(b) determining the hybridization of said probe to polynucleotides in said sample, wherein said probe comprises its sequence a region of 20 or more base pairs at least 95% identical to the polynucleotide sequence of SEQ ID NOs:2, 4, 6, 8, 10, 12, and 17.

16. A method for the treatment of a patient having need of HFGAN72 receptor comprising the step of: administering to the patient a therapeutically effective amount of the polypeptide of claim 8.

17. The method of claim 16 wherein said therapeutically effective amount of the polypeptide is administered by providing to the patient DNA encoding said polypeptide and expressing said polypeptide in vivo.

18. A process for diagnosing a disease or a susceptibility to a disease related to an under-expression of the polypeptide of claim 8 comprising the step of:
determining a mutation in a nucleic acid sequence encoding said polypeptide.

19. A diagnostic process comprising:
analyzing for the presence of the polypeptide of claim 8 in a sample derived from a host.

20. A method of detecting presence of or absence of variations in HFGAN72 receptor polynucleotides in an individual from that of a polynucleotide sequence selected from the group consisting of: SEQ ID NOs:1-18, comprising the step of:
comparing the HFGAN72 receptor polynucleotide sequences of the individual with a polynucleotide sequence selected from the group consisting of: SEQ ID NOs:1 - 18.

21. An isolated polynucleotide comprising a nucleotide sequence selected from the group consisting of:
(a) a polynucleotide sequence having at least a 95% identity to a nucleotide sequence encoding the polypeptide expressible from the cDNA insert deposited at the ATCC with Deposit Number 98806; and
(b) a nucleotide sequence complementary to the nucleotide sequence of (a).

22. An isolated polynucleotide sequence comprising the variant HFGAN72 receptor genomic polynucleotide sequence set forth in SEQ ID NOs:21 and 22.

23. An isolated polypeptide sequence comprising the variant HFGAN72 amino acid sequence set forth in SEQ ID NO:24.


SEQUENCE LISTING (24 sequences)

SEQ ID NO: 1 - DNA, 663 bp, HOMO SAPIENS
ggatgctccccttgctcgcaaggggtcgtcagtccctccgcgcaccacccccactgtgtgtgtgttgtatgtgtgcgtgtccgcgagtgcccaccggagggtctggcaggtatggcgggtggggcttgggtctctaacacctcccttggcccgttttcccaacccaaagttaaagcctctgaactggctcaagaaatatttgcaatcgggatggcttctcctcccaaatctacggtgtttggtgggttcgaacagacctggcttaagagctttgtgaccttgagcaagagactcaatctctctgagcttcggtctcatctgcaaagcagggtaccctaataatggtaaccggaaacgtccccgaaactaccttctcgtaccaggttcttggtgaagcacttggcacgcatcggagctcattactcctcatcgtggtcctgtaaggtatgtagggctgtcaccccattagacagatggggaaaccaaggctgaaagaggccaggtaagctacccaaggcaactggtgtggaattgggatgcaacccaggtctgtcttcctccaccaatttcatgactgtgagaattaagagggaacttatacgcaaagcgcctggcacaatccctaatgtttccttccttctctcttttcccactccctccttt

SEQ ID NO: 2 - DNA, 356 bp, HOMO SAPIENS
ccttcctcccttcaggaagtttgaggctgagacccgaaaagacctgggtgcaagcctccaggcaccctgaagggagtgggctgagggctggcccaagctccctcctctccctctgtagagcctaggatgcccctctgctgcagcggctcctgagctcatggagccctcagccaccccaggggcccagatgggggtcccccctggcagcagagagccgtcccctgtgcctccagactatgaagatgagtttctccgctatctgtggcgtgattatctgtacccaaaacagtatgagtgggtcctcatcgcagcctatgtggctgtgttcgtcgtggccctggtgggcaacacgctgg

SEQ ID NO: 3 - DNA, 140 bp, HOMO SAPIENS
gtaggtccagggcttgcccggcagtgctgccggctttccctggggattgaagggggttgtgtgggaggagggctcgctgattaggcagaactaggatgggtgtggctctgccaccagcttcacctcgctgcaccctgcag

SEQ ID NO: 4 - DNA, 179 bp, HOMO SAPIENS
tctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacag

SEQ ID NO: 5 - DNA, 1133 bp, HOMO SAPIENS
gtgagctctgcccaggcacccctcaccactccttgtcacgcctgtaaaaaacccacggccttgcataggtctcagtgacccccagacttgcctttcagacaggtcagtggctcatgacccctgaagtgtcatcctctgctgctagcaagggcaagccaccagatcagacactcgaggacacagacacaaccccacacactcacagagatcccctcctggtcacagccacagacatatacatagacacgtgtggacatgtatagtcacctccaggtacacagggcacacagtcaaggagagaggcaacagcccacagtgacacatacacgacaccctaggcctgctccccaatcccaaaggggcagacgtgaggggcctgatggaaacagccgtctcctctccctcctgcactggccaggaaagaccccagtggtggaaaccaggatgtccggatggggttagtggggtggaaggaaggcttctctcagtttgtatcctgtgatccacttcctgcaccccagagggcagggggcacccctagaggcaatgcccacacacctctgacccagactcatctctgcctcccagaatgagggctttttcctaacagcctggggaagggatggcattccatggcagagataaatgcctcttggatttcccactattttgaggctccccactcaactggttaactctggtgaccctgagcataaagacagatggatgagggaattctgtgcctcagtttcctcatctgtaacaggggggcaagagcgctttgtgggattgtcatgaggatgatgagaacagtgcccagcacatagtaagttacgtaggtgcaagttattattcaccggaggggtgcacctaccatgtgccaggtctaaagctggacattgtttgtacatgatttcacttattcgcacaagaatcttgccaggtagatggtatattcccattctgtagacgaggctcagagagggtgagtgacttgcccacatttacacagccagtaagtggtggagtcaggatttgcactgccctgcacctgccgtcagcctcctcactcacctactctcacatcgctgggtggcccccaaaatgaccgacgttgtgtccccgtggggcag

SEQ ID NO: 6 - DNA, 244 bp, HOMO SAPIENS
gctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcag

SEQ ID NO: 7 - DNA, 390 bp, HOMO SAPIENS
gtaatggtggaagcctcaagcaggcatcccctcaggtgggcactttgggagcacgtacccctaggacaggcatctagcagggtcccttccaaagtgggaaatcccagagcaggtatttccctaggggacaccctagactggctcctaccagggatactcccagggtgggtgcctcccctcatgtagacatctgctctagtgtagatgtccttccaggagggacaacccaagttggacaactccagggtctctgtctgtcatggtggctgtatggggtccagctgctcctaggccttgctttggccgtagtcaggacagggtggcattgctaaccagggcagggtggggctcacggattgggcctgactctgcatctcttgacccctgcag

SEQ ID NO: 8 - DNA, 116 bp, HOMO SAPIENS
atgacctctatcccaagatctaccacagttgcttctttattgtcacctacctggccccactgggcctcatggccatggcctatttccagatattccgcaagctctggggccgccag

SEQ ID NO: 9 - DNA, 1929 bp, HOMO SAPIENS
gtgaggcccactctgggcaggggctaggccagtcactgtgtgggctgggggtgggagggctactggtctaactgagtaggcagtcctctgccatcagcacatgccatcttggctgcaaccaaagagaggggaagcccagagacacgtcaaactcaaggccaaaagcaccagtggctaccctggaatggaatagtaacacgtccttctattagtggttggcgtttattgaagtatccactcccagataatcttgcatcctcttagccaccatatattacccacattaaatatatgagaaaaccgagacccagaagatcaacataacttcccccaaaccactcaggtagtgagtagatcaggaactaaagcccagatctgtgagctcccactgctcagtttagtaccactgcaacaataataatagcaactccgtggtgcttgccaaattaggcactttgcatccaatgtcttaacaactatctaacaaaagaagcaacattacccacgtcacaaatgccaaataagggcaaccaacttgccagattcaacagcagcagagccttctggttccagggcctgtcttctttcctgcattacagactgacccacggtgggtttcttaggtttttggggggcaggggtggtcagaggcccttggcctagcgagtgggagtcctggattggcgtctgggcggtgagaaaaggcaggccagaacatgaccaggctcaggaagggactctcacacttggggatgtcacctacattccacaggaagtactggcttgcacccaggccatgccgggcagcggatggggacacggactggctgtgacccaggtcctgccttggaggagcacccagtccagtaggacccttcctgactggccagccctgtagtccaccaacactcatcatctgctccccacagaccccccagccaagcaggacacaggcacgatcctcctcatttgacagatatgaaagcaaggcttagaaaggaaaatgaggtggctaaggtcacatagctcacgaatggctgagctggctctagacccgcgtttccagaactccagccccatgcccctctgtggtgggtgatttgagtgtccggtggcaggagaggcttctccaggagcccagaaccaccccaggcttatgggcactggcccaggccattcgatgctgcccacctgctcaccccttgcccaggcctcctcatagtctggtatgatccaggggaggcacaactcacccccacccctaccctcaaagatagtgttggagatttagggaggatggatgggcagttgacaggatgtggcctggggtcttgtcaaggttccccacctctttgagtcttagttgcctcatctatacctaaggaccaataatatctttccacaaggcgtgttgtagagggtttcacaaagagctaatggaaaatgaaagtctaggctgggcgcagtggctcacacctgtattcccagcactttgggaggctgaggcaggcggatcacctaaggtcaggagttcaagaccagcctggccaacgtggtgaaaccccatctctactaaaaatacaaaacttagcccggtgtggtggcgcacacctgtaatcccagctactcgggaggcgagattgaagagagccaagattgcaccattgcactccagcttaggtgacaagagtgaaatgccatctcaaaaaaaaaaaaaaagaaaagaaaagaaaatgaaagtctatcgttcactctcaagtccagagtgttagtctatcataaacattagattccttcctcttgcaagggttttatccttttgcccatctccaccctgcccggggtccagcctggagtaggccccacaaaaggcaaccaccctcccaaggtgctgtacccaccactgctgtctctatgtgtgctggacag

SEQ ID NO: 10 - DNA, 227 bp, HOMO SAPIENS
atccccggcaccacctcagcactggtgcggaactggaagcgcccctcagaccagctgggggacctggagcagggcctgagtggagagccccagccccgggcccgcgccttcctggctgaagtgaagcagatgcgtgcacggaggaagacagccaagatgctgatggtggtgctgctggtcttcgccctctgctacctgcccatcagcgtcctcaatgtccttaagag

SEQ ID NO: 11 - DNA, 1248 bp, HOMO SAPIENS
gtgagagcacggggtatggttggggtggggagaagtttgaggttggggaaggagctctccttgcttgggagaaagacctggctccaccccttctccactatgtgatcttgggcaggccatttctcttctctgagcctccatctcctagggctatcgtgaaaattcacgcattcattcacttaatcatcacattttagggggctggaaatacaatgaacaagtgcataagacagacaaagtccctgccttcatggaggctgcattctagcaggagagaagggaagtaaatagaagaatcaatgtatattataatgtcaggcagtgataactgctgggaagaaaaataaaataggacagagagtgacaatgataagggttggtgggtttttgcttttgctttagatacaatggtttaaaaaaagcagggggccgggtgcagtggctcacatctgtaatcccaacacgttgggaggccaaggagggaggatcgcttgaggccaggagttcaagatcagcccgggcaacataatgagacttcgtctctactaaaattcaaaaaattagccagccatggtggcatgtgcctgtagttctagctacacagactgaggtggaagaatagcttgagcccaggaggttgaggctgcagcgaaccatgattgcaccactgcactccagcctgggtgacacagctgtctcaaaaaaaaaaaaaaaaaaaaaaaagcctttccaaggaaatgacatttgagcagagacttgaaggaagtgagagagctaaccatgcacgtgtctgtagggacagccaaagagggtcgcagggcgctggggagagaatgcaggctattggacagaagacagtttcactttgagattgtgcttggccacttcctggttgtgtgatcttcggcatgtcactttacttctctgagcctcagtttccttaatggaaaaatggatgatgtctatgattcatcatgttgctgtgaggatggatgagaaagtggatgggaagccccaggggatccgatggccaggaggctagagatgcccatcacggtgcttgataccctccatgcttgagaaccccaaaccctggccaagacctcaggtacagaaggccaggaaacgtggacagaagtgggcagtaggaactcttgcactttacagctcaggttctgtgagcagcactcccccagtacatgcatacgcagctaccccatttctgacgctcctccaccctgggcctag

SEQ ID NO: 12 - DNA, 122 bp, HOMO SAPIENS
ggtgttcgggatgttccgccaagccagtgaccgcgaagctgtctacgcctgcttcaccttctcccactggctggtgtacgccaacagcgctgccaaccccatcatctacaacttcctcagtg

SEQ ID NO: 13 - DNA, 1672 bp, HOMO SAPIENS
gtgagcaggctggggatgcaaaatgactgagggtggccaacagtccacatgacaagtctccccatccccaagccagggcccaaataaaggatggtgggtgaggatgtacctgctgtgggcacagtgatcctgctctgggaggacccaccccaagcggccctggcctgagtgggagacgggccacactccctacagtggctggcacccaggatccagttttgcagattctgcagaccagtgagtgagtggaagggcaggggctaggccagctcacccccaactcccaccctgggtgcaggcacagcaagacctccaatcagctcaggcagaggagtccatcctccccggagggagtcagacctgtgggaggagggccctggagcccctgcccgaggaaggattgcacagtccaggtgtcagggctaaagtagggtcactctgagagacaagccaggcccagggaagggcttcgccggctcagctagacacactggcagagtgaccggaatctcaggggttgtcccctctggaagtcttcctcccctgccacccccactcccactccaggcctctcctctctgctgtcccacagtgcccaccccctccctctacctcccagtctcagggtggtaatggctctgaggctgagctcagcagaagtctgactcaccacccctctgactttgggaatagacttctaaagaacaggtccagatgactgttgaagcctggacagaaataatctttgaggaactattaaaaggttaaagaaaggatcaggagtcaatagtataaccctcattgagactcaagaattactcaacaaggctggctgcgggtttccaggtcagaaaagagaataaatgatgacctgtgtggggaggggagggcaaacaaacttactgacacatatgcctttgtttggcctatgtttactgagcacctactatgtgcttgaccctgtgctgggcaccagagaggctggcagcctaatgacacatgatcaaaggggcttcagcctgacaaaatctgtttccctggtatacttgggctgaataatgtggtgtggtggtccctccttccctcctcccccttgagaagggctttggaattagaattgggttcagcttctggctgggtggacttgggcaagccactgtacctctgtgcatctcatctgtgaagtgaggataaaggactccagcctttcagggtgctgggatgctctggcggacagaggctgaggcgcccagcacagcgtgactgccaaatgcaaaagggctgctgctgccgtcattttcatcatcaaagggcagagaggacacaagcctcgcaacagatagtgacccccacgtacacaccaagggagagcagaggtgacctgaggcccccgagccagacaccacgttttgagtcagcctccgagccagagcacagtcaaggaatcagatggcaattgcgtctctccttgggaacccgctccagggcttctgtcctctctctctggcggtgccgaggttgcctcagggctctccctcccagctctatccctccctccctccccgccccctcataggcagcttggctggagctgcgtgggtgtccctgggctcaaggccccttcctgctgcatctgtctccttatggctgtgtcttttgtctcccaaccaag

SEQ ID NO: 14 - DNA, 324 bp, HOMO SAPIENS
gcaaattccgggagcagtttaaggctgccttctcctgctgcctgcctggcctgggtccctgcggctctctgaaggcccctagtccccgctcctctgccagccacaagtccttgtccttgcagagccgatgctccatctccaaaatctctgagcatgtggtgctcaccagcgtcaccacagtgctgccctgagcgagggctgccctggaggctccggctcgggggatctgcccctacccctcatggaaagacagctggatgtggtgaaaggctgtggcttcagtcctgggtttctgcctgtgtgactctggataagtcacttcct

SEQ ID NO: 15 - DNA, 1710 bp, HOMO SAPIENS
ctgtctgagcttgtgtcccctaagcagggttgatgtgaggattaagcatgctgaagcaagtggaaagctccttgtaaactgtgaagtgttgtggacatgattattgttgtacttctctcatttggccataccccacagtataatctgtccccatcctccttccagagcttggtcatcctcctaaagacccctttcctacccaattacaggccttccctggagtctgctctaaaggtcccaacaggcatttccatcttgttccatggctccctgaagcccagggctgcacttggccagctgttctgatgcctgtgtgaactaatctgggcccagcctttctccagcgggccacgagcacagccccaccctaaccaggtgccaagggcacacaccacagacccgaccttgttggctttgtggtgtgataaaacactctccatggccacttggcagagaggccagcagcccgaagcaactgtaattaaaagcctggcactgaatgttccctttccttgtcattgcacaaaatctgtgctgcttaggttaggagcagaagaaggtggggaagctggggggagggaagacaagaaggcactagaatgctcttatctgctgacctcccacccccaccacacgggatcccagatggactcagccaggcctcggcacacgtggtgctgcctcctcatgcggtgccaatcctgtgacgccgggacctcacccctcgacacaactgtctgctcacaggcccagtggcctggggaaggtaaggggggccgggtgggcagatagcgggttaacaggctgatttactgttatcttcatcttcttggaaaccaaggcttgcgcctattttctatgaactcacaggataaaaagagaaagccacagacttggccagacatgctccgtctgccaccgagattttaaggctgccaaaggccctgagccttgttgccagagaaggacgtggagtgcagcctgaagtgctactctgctgagaggaacagggacatctggcaggggccagctctgctatgcataccaccaggtctgcttaacttgattaatagagctgtgggctccctttgacctcaaccgtcaagaacgtgggcagagccctggtgctgccaggggcccagcccaggagtgttgaagcactgctcctcctaagcaccccgctttacccagagcaggtggaatgtgtggtttaagtatgccacctgctctccacacgacttaccagcccagctctctgtggacggggcagagctctagctgctgccaggaggatgggctctcctgcccattccacactcccactggccttaggatccaaagcggttggcacagtgcataggtaggcttggaggtgagtgtgcagaatgaggtatgagggctggcccccaagctcagaaccaaccgtgcttacaagatcaatccaggtcttgggctcctcagcctcagctgagcacaaaggtgagagactacaattggcaccatcgggtttcagcagacacttggttcagtgggaacaaagaaacaacaggcgctgctcctaatggctgcacgctctgctggctgaacccagcaccatggggggcctcagcccttcactttgggggactcagataaactgaagaaaattcaggggaggtgaccaggatgccaagggcagagatgctcagcgagggactgctcaagagccca

SEQ ID NO: 16 - DNA, 1265 bp, HOMO SAPIENS
gagctcccaggaccaccttgcgtggggcagacatcagaggcagtacagcagaggtatgaggacctgttctggagtcagccactgggcaagttgcttagcctcactatccctattttatcatctctaaaatggggataatacttaacttgtaggttctagtaaggattaaaacgggtgatcatgtaaagcacttagcacagtgactggcacagaaaaagcacttggtaaatgctattttaataaagtcactgtcactccaaggtctcttggagcatcctgtctcttcactgggatgcttccttccttcctctgttcagaacttaccaggccaggctaaaacctcagctctccatttgttatgcgggcccttgggcaggttacctacactaaacctcagttttctcaccggtaaaagggagattataagggattcttcattgaagggtgtggaattataaatagtacctagcacatgggaagaatgaattacataagcctattaaaaatagcaagcattcagtaaatacgttttgttgtggttaatatcacccttactaatatccacgacaaaagctggcaagagtaggtgattactataggtcatctcccctcccttttactgggggcagatgtggtgcactcactcctgcccacagacctccttagtctgaatgaaatccacagcctgggcaaaggactagactctctggggagccctttctccctcctgggtggcaaagctccctcttgggtcctcccccaacctcttactaacctgcctgagcccaaactgtggccttctgccttacacagtcagtttggaaccagacctggggctgtggcctcaggtcatcttccccaactgcaacttttagagtgctcagggtcccaaaagttgggaaagaaactgtccactagcagaggtggcaacagacccatctttctgagccctgttcagggctctttccattctacatgctagaaactggagagaaaggagtcacccaagccacacacactggtctcatagtccttaaggtgccaagtttccatccttaaggctccctgtggctggggccgtctcacccagcactccagggtccagccctgattctggacccaggtgtttccttgccctggctcttggctcagtgccccagctgggtcaaagaaggggtgagggatgcaggcctgcactgagaacagcattttgtagtgcaggtggacattcggtaacaagaggaaacccctttgttgtatcttag

SEQ ID NO: 17 - DNA, 96 bp, HOMO SAPIENS
ggtgtaaagagaagagtctagctctgtcctgcccatcgtgccccggccatgacccgcaccttgctgcagctctgtgtagctaatggagcccgagcg

SEQ ID NO: 18 - DNA, 467 bp, HOMO SAPIENS
gtcccggtcatactgctggaagaggttcttccactgctggatgaatttccacagggctgagaagccgtagacatcgatgcggcctgacttggtcttgtcaaacatgtctgaaggtgaggagggaaaggaggggaggaaggcttcaagactgaaggccaagggtccccctcaggacccattgaggcagcccttctctccaggctggagtaggcgacttcacagaaccaagccctgaggcccagatgcctgcacctgagacacccacatcaagcgttgcaccgtcctcctattctggacacctttagccccaccccatccagaactgcattttctctgcaggcttggcacctgacccctccctgctgtttcctacaggggagggagtaccattctacagaagaggaaactaagttaacctgtaactaggcagggtctagaacttaagaaagatctcaggaaaagagctc

SEQ ID NO: 19 - protein, 425 aa, HOMO SAPIENS
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 20 - protein, 389 aa, HOMO SAPIENS
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGCKEKSLALSCPSCPGHDPHLAAALCS

SEQ ID NO: 21 - DNA, 10453 bp, HOMO SAPIENS
ggatgctccccttgctcgcaaggggtcgtcagtccctccgcgcaccacccccactgtgtgtgtgttgtatgtgtgcgtgtccgcgagtgcccaccggagggtctggcaggtatggcgggtggggcttgggtctctaacacctcccttggcccgttttcccaacccaaagttaaagcctctgaactggctcaagaaatatttgcaatcgggatggcttctcctcccaaatctacggtgtttggtgggttcgaacagacctggcttaagagctttgtgaccttgagcaagagactcaatctctctgagcttcggtctcatctgcaaagcagggtaccctaataatggtaaccggaaacgtccccgaaactaccttctcgtaccaggttcttggtgaagcacttggcacgcatcggagctcattactcctcatcgtggtcctgtaaggtatgtagggctgtcaccccattagacagatggggaaaccaaggctgaaagaggccaggtaagctacccaaggcaactggtgtggaattgggatgcaacccaggtctgtcttcctccaccaatttcatgactgtgagaattaagagggaacttatacgcaaagcgcctggcacaatccctaatgtttccttccttctctcttttcccactccctcctttccttcctcccttcaggaagtttgaggctgagacccgaaaagacctgggtgcaagcctccaggcaccctgaagggagtgggctgagggctggcccaagctccctcctctccctctgtagagcctaggatgcccctctgctgcagcggctcctgagctcatggagccctcagccaccccaggggcccagatgggggtcccccctggcagcagagagccgtcccctgtgcctccagactatgaagatgagtttctccgctatctgtggcgcgattatctgtacccaaaacagtatgagtgggtcctcatcgcagcctatgtggctgtgttcgtcgtggccctggtgggcaacacgctgggtaggtccagggcttgcccggcagtgctgccggctttccctggggattgaagggggttgtgtgggaggagggctcgctgattaggcagaactaggatgggtgtggctctgccaccagcttcacctcgctgcaccctgcagtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggtgagctctgcccaggcacccctcaccactccttgtcacgcctgtaaaaaacccacggccttgcataggtctcagtgacccccagacttgcctttcagacaggtcagtggctcatgacccctgaagtgtcatcctctgctgctagcaagggcaagccaccagatcagacactcgaggacacagacacaaccccacacactcacagagatcccctcctggtcacagccacagacatatacatagacacgtgtggacatgtatagtcacctccaggtacacagggcacacagtcaaggagagaggcaacagcccacagtgacacatacacgacaccctaggcctgctccccaatcccaaaggggcagacgtgaggggcctgatggaaacagccgtctcctctccctcctgcactggccaggaaagaccccagtggtggaaaccaggatgtccggatggggttagtggggtggaaggaaggcttctctcagtttgtatcctgtgatccacttcctgcaccccagagggcagggggcacccctagaggcaatgcccacacacctctgacccagactcatctctgcctcccagaatgagggctttttcctaacagcctggggaagggatggcattccatggcagagataaatgcctcttggatttcccactattttgaggctccccactcaactggttaactctggtgaccctgagcataaagacagatggatgagggaattctgtgcctcagtttcctcatctgtaacaggggggcaagagcgctttgtgggattgtcatgaggatgatgagaacagtgcccagcacatagtaagttacgtaggtgcaagttattattcaccggaggggtgcacctaccatgtgccaggtctaaagctggacattgtttgtacatgatttcacttattcgcacaagaatcttgccaggtagatggtatattcccattctgtagacgaggctcagagagggtgagtgacttgcccacatttacacagccagtaagtggtggagtcaggatttgcactgccctgcacctgccgtcagcctcctcactcacctactctcacatcgctgggtggcccccaaaatgaccgacgttgtgtccccgtggggcaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcaggtaatggtggaagcctcaagcaggcatcccctcaggtgggcactttgggagcacgtacccctaggacaggcatctagcagggtcccttccaaagtgggaaatcccagagcaggtatttccctaggggacaccctagactggctcctaccagggatactcccagggtgggtgcctcccctcatgtagacatctgctctagtgtagatgtccttccaggagggacaacccaagttggacaactccagggtctctgtctgtcatggtggctgtatggggtccagctgctcctaggccttgctttggccgtagtcaggacagggtggcattgctaaccagggcagggtggggctcacggattgggcctgactctgcatctcttgacccctgcagatgacctctatcccaagatctaccacagttgcttctttattgtcacctacctggccccactgggcctcatggccatggcctatttccagatattccgcaagctctggggccgccaggtgaggcccactctgggcaggggctaggccagtcactgtgtgggctgggggtgggagggctactggtctaactgagtaggcagtcctctgccatcagcacatgccatcttggctgcaaccaaagagaggggaagcccagagacacgtcaaactcaaggccaaaagcaccagtggctaccctggaatggaatagtaacacgtccttctattagtggttggcgtttattgaagtatccactcccagataatcttgcatcctcttagccaccatatattacccacattaaatatatgagaaaaccgagacccagaagatcaacataacttcccccaaaccactcaggtagtgagtagatcaggaactaaagcccagatctgtgagctcccactgctcagtttagtaccactgcaacaataataatagcaactccgtggtgcttgccaaattaggcactttgcatccaatgtcttaacaactatctaacaaaagaagcaacattacccacgtcacaaatgccaaataagggcaaccaacttgccagattcaacagcagcagagccttctggttccagggcctgtcttctttcctgcattacagactgacccacggtgggtttcttaggtttttggggggcaggggtggtcagaggcccttggcctagcgagtgggagtcctggattggcgtctgggcggtgagaaaaggcaggccagaacatgaccaggctcaggaagggactctcacacttggggatgtcacctacattccacaggaagtactggcttgcacccaggccatgccgggcagcggatggggacacggactggctgtgacccaggtcctgccttggaggagcacccagtccagtaggacccttcctgactggccagccctgtagtccaccaacactcatcatctgctccccacagaccccccagccaagcaggacacaggcacgatcctcctcatttgacagatatgaaagcaaggcttagaaaggaaaatgaggtggctaaggtcacatagctcacgaatggctgagctggctctagacccgcgtttccagaactccagccccatgcccctctgtggtgggtgatttgagtgtccggtggcaggagaggcttctccaggagcccagaaccaccccaggcttatgggcactggcccaggccattcgatgctgcccacctgctcaccccttgcccaggcctcctcatagtctggtatgatccaggggaggcacaactcacccccacccctaccctcaaagatagtgttggagatttagggaggatggatgggcagttgacaggatgtggcctggggtcttgtcaaggttccccacctctttgagtcttagttgcctcatctatacctaaggaccaataatatctttccacaaggcgtgttgtagagggtttcacaaagagctaatggaaaatgaaagtctaggctgggcgcagtggctcacacctgtattcccagcactttgggaggctgaggcaggcggatcacctaaggtcaggagttcaagaccagcctggccaacgtggtgaaaccccatctctactaaaaatacaaaacttagcccggtgtggtggcgcacacctgtaatcccagctactcgggaggcgagattgaagagagccaagattgcaccattgcactccagcttaggtgacaagagtgaaatgccatctcaaaaaaaaaaaaaaagaaaagaaaagaaaatgaaagtctatcgttcactctcaagtccagagtgttagtctatcataaacattagattccttcctcttgcaagggttttatccttttgcccatctccaccctgcccggggtccagcctggagtaggccccacaaaaggcaaccaccctcccaaggtgctgtacccaccactgctgtctctatgtgtgctggacagatccccggcaccacctcagcactggtgcggaactggaagcgcccctcagaccagctgggggacctggagcagggcctgagtggagagccccagccccgggcccgcgccttcctggctgaagtgaagcagatgcgtgcacggaggaagacagccaagatgctgatggtggtgctgctggtcttcgccctctgctacctgcccatcagcgtcctcaatgtccttaagaggtgagagcacggggtatggttggggtggggagaagtttgaggttggggaaggagctctccttgcttgggagaaagacctggctccaccccttctccactatgtgatcttgggcaggccatttctcttctctgagcctccatctcctagggctatcgtgaaaattcacgcattcattcacttaatcatcacattttagggggctggaaatacaatgaacaagtgcataagacagacaaagtccctgccttcatggaggctgcattctagcaggagagaagggaagtaaatagaagaatcaatgtatattataatgtcaggcagtgataactgctgggaagaaaaataaaataggacagagagtgacaatgataagggttggtgggtttttgcttttgctttagatacaatggtttaaaaaaagcagggggccgggtgcagtggctcacatctgtaatcccaacacgttgggaggccaaggagggaggatcgcttgaggccaggagttcaagatcagcccgggcaacataatgagacttcgtctctactaaaattcaaaaaattagccagccatggtggcatgtgcctgtagttctagctacacagactgaggtggaagaatagcttgagcccaggaggttgaggctgcagcgaaccatgattgcaccactgcactccagcctgggtgacacagctgtctcaaaaaaaaaaaaaaaaaaaaaaaagcctttccaaggaaatgacatttgagcagagacttgaaggaagtgagagagctaaccatgcacgtgtctgtagggacagccaaagagggtcgcagggcgctggggagagaatgcaggctattggacagaagacagtttcactttgagattgtgcttggccacttcctggttgtgtgatcttcggcatgtcactttacttctctgagcctcagtttccttaatggaaaaatggatgatgtctatgattcatcatgttgctgtgaggatggatgagaaagtggatgggaagccccaggggatccgatggccaggaggctagagatgcccatcacggtgcttgataccctccatgcttgagaaccccaaaccctggccaagacctcaggtacagaaggccaggaaacgtggacagaagtgggcagtaggaactcttgcactttacagctcaggttctgtgagcagcactcccccagtacatgcatacgcagctaccccatttctgacgctcctccaccctgggcctagggtgttcgggatgttccgccaagccagtgaccgcgaagctgtctacgcctgcttcaccttctcccactggctggtgtacgccaacagcgctgccaaccccatcatctacaacttcctcagtggtgagcaggctggggatgcaaaatgactgagggtggccaacagtccacatgacaagtctccccatccccaagccagggcccaaataaaggatggtgggtgaggatgtacctgctgtgggcacagtgatcctgctctgggaggacccaccccaagtggccctggcctgagtgggagacgggccacactccctacagtggctggcacccaggatccagttttgcagattctgcagaccagtgagtgagtggaagggcaggggctaggccagctcacccccaactcccaccctgggtgcaggcacagcaagacctccaatcagctcaggcagaggagtccatcctccccggagggagtcagacctgtgggaggagggccctggagcccctgcccgaggaaggattgcacagtccaggtgtcagggctaaagtagggtcactctgagagacaagccaggcccagggaagggcttcgccggctcagctagacacactggcagagtgaccggaatctcaggggttgtcccctctggaagtcttcctcccctgccacccccactcccactccaggcctctcctctctgctgtcccacagtgcccaccccctccctctacctcccagtctcagggtggtaatggctctgaggctgagctcagcagaagtctgactcaccacccctctgactttgggaatagacttctaaagaacaggtccagatgactgttgaagcctggacagaaataatctttgaggaactattaaaaggttaaagaaaggatcaggagtcaatagtataaccctcattgagactcaagaattactcaacaaggctggctgcgggtttccaggtcagaaaagagaataaatgatgacctgtgtggggaggggagggcaaacaaacttactgacacatatgcctttgtttggcctatgtttactgagcacctactatgtgcttgaccctgtgctgggcaccagagaggctggcagcctaatgacacatgatcaaaggggcttcagcctgacaaaatctgtttccctggtatacttgggctgaataatgtggtgtggtggtccctccttccctcctcccccttgagaagggctttggaattagaattgggttcagcttctggctgggtggacttgggcaagccactgtacctctgtgcatctcatctgtgaagtgaggataaaggactccagcctttcagggtgctgggatgctctggcggacagaggctgaggcgcccagcacagcgtgactgccaaatgcaaaagggctgctgctgccgtcattttcatcatcaaagggcagagaggacacaagcctcgcaacagatagtgacccccacgtacacaccaagggagagcagaggtgacctgaggcccccgagccagacaccacgttttgagtcagcctccgagccagagcacagtcaaggaatcagatggcaattgcgtctctccttgggaacccgctccagggcttctgtcctctctctctggcggtgccgaggttgcctcagggctctccctcccagctctatccctccctccctccccgccccctcataggcagcttggctggagctgcgtgggtgtccctgggctcaaggccccttcctgctgcatctgtctccttatggctgtgtcttttgtctcccaaccaaggcaaattccgggagcagtttaaggctgccttctcctgctgcctgcctggcctgggtccctgcggctctctgaaggcccctagtccccgctcctctgccagccacaagtccttgtccttgcagagccgatgctccgtctccaaaatctctgagcatgtggtgctcaccagcgtcaccacagtgctgccctgagcgagggctgccctggaggctccggctcgggggatctgcccctacccctcatggaaagacagctggatgtggtgaaaggctgtggcttcagtcctgggtttctgcctgtgtgactctggataagtcacttcctctgtctgagcttgtgtccccgaagcagggttgatgtgaggattaagcatgctgaagcaagtggaaagctccttgtaaactgtgaagtgttgtggacatgattattgttgtacttctctcatttggccataccccacagtataatctgtccccatcctccttccagagcttggtcatcctcctaaagacccctttcctacccaattacaggccttccctggagtctgctctaaaggtcccaacaggcatttccatcttgttccatggctccctgaagcccagggctgcacttggccagctgttctgatgcctgtgtgaactaatctgggcccagcctttctccagcgggccacgagcacagccccaccctaaccaggtgccaagggcacacaccacagacccgaccttgttggctttgtggtgtgataaaacactctccatggccacttggcagagaggccagcagcccgaagcaactgtaattaaaagcctggcactgaatgttccctttccttgtcattgcacaaaatctgtgctgcttaggttaggagcagaagaaggtggggaagctggggggagggaagacaagaaggcactagaatgctcttatctgctgacctcccacccccaccacacgggatcccagatggactcagccaggcctcggcacacgtggtgctgcctcctcatgcggtgccaatcctgtgacgccgggacctcacccctcgacacaactgtctgctcacaggcccagtggcctggggaaggtaaggggggccgggtgggcagatagcgggttaacaggctgatttactgttatcttcatcttcttggaaaccaaggcttgcgcctattttctatgaactcacaggataaaaagagaaagccacagacttggccagacatgctccgtctgccaccgagattttaaggctgccaaaggccctgagccttgttgccagagaaggacgtggagtgcagcctgaagtgctactctgctgagaggaacagggacatctggcaggggccagctctgctatgcataccaccaggtctgcttaacttgattaatagagctgtgggctccctttgacctcaaccgtcaagaacgtgggcagagccctggtgctgccaggggcccagcccaggagtgttgaagcactgctcctcctaagcaccccgctttacccagagcaggtggaatgtgtggtttaagtatgccacctgctctccacacgacttaccagcccagctctctgtggacggggcagagctctagctgctgccaggaggatgggctctcctgcccattccacactcccactggccttaggatccaaagcggttggcacagtgcataggtaggcttggaggtgagtgtgcagaatgaggtatgagggctggcccccaagctcagaaccaaccgtgcttacaagatcaatccaggtcttgggctcctcagcctcagctgagcacaaaggtgagagactacaattggcaccatcgggtttcagcagacacttggttcagtgggaacaaagaaacaacaggcgctgctcctaatggctgcacgctctgctggctgaacccagcaccatggggggcctcagcccttcactttgggggactcagataaactgaagaaaattcaggggaggtgaccaggatgccaagggcagagatgctcagcgagggactgctcaagagccca

SEQ ID NO: 22 - DNA, 1828 bp, HOMO SAPIENS
gagctcccaggaccaccttgcgtggggcagacatcagaggcagtacagcagaggtatgaggacctgttctggagtcagccactgggcaagttgcttagcctcactatccctattttatcatctctaaaatggggataatacttaacttgtaggttctagtaaggattaaaacgggtgatcatgtaaagcacttagcacagtgactggcacagaaaaagcacttggtaaatgctattttaataaagtcactgtcactccaaggtctcttggagcatcctgtctcttcactgggatgcttccttccttcctctgttcagaacttaccaggccaggctaaaacctcagctctccatttgttatgcgggcccttgggcaggttacctacactaaacctcagttttctcaccggtaaaagggagattataagggattcttcattgaagggtgtggaattataaatagtacctagcacatgggaagaatgaattacataagcctattaaaaatagcaagcattcagtaaatacgttttgttgtggttaatatcacccttactaatatccacgacaaaagctggcaagagtaggtgattactataggtcatctcccctcccttttactgggggcagatgtggtgcactcactcctgcccacagacctccttagtctgaatgaaatccacagcctgggcaaaggactagactctctggggagccctttctccctcctgggtggcaaagctccctcttgggtcctcccccaacctcttactaacctgcctgagcccaaactgtggccttctgccttacacagtcagtttggaaccagacctggggctgtggcctcaggtcatcttccccaactgcaacttttagagtgctcagggtcccaaaagttgggaaagaaactgtccactagcagaggtggcaacagacccatctttctgagccctgttcagggctctttccattctacatgctagaaactggagagaaaggagtcacccaagccacacacactggtctcatagtccttaaggtgccaagtttccatccttaaggctccctgtggctggggccgtctcacccagcactccagggtccagccctgattctggacccaggtgtttccttgccctggctcttggctcagtgccccagctgggtcaaagaaggggtgagggatgcaggcctgcactgagaacagcattttgtagtgcaggtggacattcggtaacaagaggaaacccctttgttgtatcttagggtgtaaagagaagagtctagctctgtcctgcccatcgtgccccggccatgacccgcaccttgctgcagctctgtgtagctaatggagcccgagcggtcccggtcatactgctggaagaggttcttccactgctggatgaatttccacagggctgagaagccgtagacatcgatgcggcctgacttggtcttgtcaaacatgtctgaaggtgaggagggaaaggaggggaggaaggcttcaagactgaaggccaagggtccccctcaggacccattgaggcagcccttctctccaggctggagtaggcgacttcacagaaccaagccctgaggcccagatgcctgcacctgagacacccacatcaagcgttgcaccgtcctcctattctggacacctttagccccaccccatccagaactgcattttctctgcaggcttggcacctgacccctccctgctgtttcctacaggggagggagtaccattctacagaagaggaaactaagttaacctgtaactaggcagggtctagaacttaagaaagatctcaggaaaagagctc

SEQ ID NO: 23 - DNA, 1564 bp, HOMO SAPIENS
cctcccttcaggaagtttgaggctgagacccgaaaagacctgggtgcaagcctccaggcaccctgaagggagtgggctgagggctggcccaagctccctcctctccctctgtagagcctaggatgcccctctgctgcagcggctcctgagctcatggagccctcagccaccccaggggcccagatgggggtcccccctggcagcagagagccgtcccctgtgcctccagactatgaagatgagtttctccgctatctgtggcgcgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcacggaggaagacagccaagatgctgatggtggtgctgctggtcttcgccctctgctacctgcccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggcaaattccgggagcagtttaaggctgccttctcctgctgcctgcctggcctgggtccctgcggctctctgaaggcccctagtccccgctcctctgccagccacaagtccttgtccttgcagagccgatgctccgtctccaaaatctctgagcatgtggtgctcaccagcgtcaccacagtgctgccctgagcgagggctgccctggaggctccggctcgggggatctgcccctacccctcatggaaagacagctggatgtggtgaaaggctgtggcttcagtcctgggtttctgcctgtgtgactctggataagtcacttcct

SEQ ID NO: 24 - protein, 425 aa, HOMO SAPIENS
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSVSKISEHVVLTSVTTVLP